(19) United States
(12) Patent Application Publication
(10) Pub. No.: US 2008/0057522 A1
(43) Pub. Date: Mar. 6, 2008

(54) Method for Screening Agents Modulating Ikbalpha Protein Ubiquitination and Means for Carrying out Said Method

(76) Inventors: Cecile Bougeret, Gif Sur Yvette (FR); Patrick Zarzov, Viroflay (FR); Jean-Francois Briand, Paris (FR); Dominique Thomas, Gif Sur Yvette (FR)

Correspondence Address: YOUNG & THOMPSON, 745 SOUTH 23RD STREET, 2ND FLOOR, ARLINGTON, VA, 22202, US

(21) Appl. No.: 10/592,944

(22) PCT Filed: Mar. 15, 2005
(86) PCT No.: PCT/FR05/50165

§ 371 Date: Sep. 15, 2006

(30) Foreign Application Priority Data
Mar. 16, 2004 (FR) 0450528

(51) Int. Cl. G01N 33/53; C07H 21/00; C12N 1/19; C12N 15/63; C12Q 1/02; C12Q 1/34; C12Q 1/66

(52) U.S. Cl. 435/7.31; 435/18; 435/254.2; 435/29; 435/320.1; 435/8; 536/23.2; 536/23.4

(57) ABSTRACT

A method for screening agents modulating IκBα protein ubiquitination by a functional ubiquitin ligase protein complex containing β-TrCP protein, the method including the following steps:

(a) bringing into contact a candidate agent to be tested with recombinant yeast cells that express in their nucleus:
(i) a fusion protein including the IκBα polypeptide and at least one first detectable protein; and
(ii) a protein containing the β-TrCP polypeptide;

(b) quantifying the first detectable protein in the yeast cells, at the end of at least one predetermined period of time after bringing the candidate agent into contact with the cells;

(c) comparing the result obtained in step (b) with a control result obtained when step (a) is carried out in the absence of the candidate.

### FIELD OF THE INVENTION

The present invention concerns the screening of biologically active agents able to modulate IκBα protein ubiquitination, particularly therapeutic agents of therapeutic interest, and more specifically therapeutic agents directed to preventing or treating inflammatory or autoimmune diseases or cancers.

### STATE OF THE ART

One of the major unresolved medical problems is the development of effective treatments for inflammatory and autoimmune syndromes. These pathologies are currently treated using non-steroid anti-inflammatory drugs such as aspirin and ibuprofen, and corticosteroids, which are of limited efficacy and have considerable toxic side effects. The most specific cyclooxygenase inhibitors, such as refecoxib and tumour necrosis factor (TNF) blocking agents, which have appeared on the market more recently, have proved to have the same disadvantages.

Transcription factors of the NF-κB family form part of the body's first line of defence against viral, bacterial or fungal infections and also in situations of physiological stress. These transcription factors determine the expression of a large number of genes, including many genes coding for inflammation mediators. These include genes coding for the TNF-α factor, IL-1,IL-6 and IL-8 interleukins, adhesion molecules ICAM-1, VCAM-1 and E-Selectin, NO-synthase and Cox2 prostaglandin synthase.

The factors of the NF-κB family are activated by a large number of endogenous and exogenous pathogenic stimuli, including bacterial lipids or proteins, cytokines, growth factors and molecules linked to oxidative stress situations. Activation of NF-κB factors, in response to these pathogenic stimuli, is observed for almost all cells involved in immune response, such as epithelial cells, mesenchyme cells, lymphocytes, neutrophils and macrophages.

Today, although the exact aetiology of most chronic inflammatory syndromes has still not been determined, experimental results, including the results of clinical studies, have shown the important role played by activation of the NF-κB factor, both in initiating inflammation and in establishing a chronic inflammatory state. Thus, blocking activation of factors belonging to the NF-κB family constitutes an effective pathway to treat inflammatory syndromes such as asthma, rheumatoid arthritis, inflammatory colopathies such as Crohn's disease, multiple sclerosis and psoriasis (Ballard, 2001; Baud and Karin, 2001).

It has now been established that the inflammatory response and activation of the NF-κB factor is directly linked to the destruction of the IκBα factor by the ubiquitin proteasome system (Kroll et al, 1999; Winston et al, 1999). Indeed, in non-stimulated or non-activated cells the NF-κB factor is sequestered in the cell cytoplasm. So, in non-stimulated or non-activated cells, the NF-κB factor is incapable of activating the expression of the target genes for this factor. The activation of the target genes first needs translocation of the factor NF-κB from the cytoplasm to the nucleus. This translocation is triggered by the degradation of the IκBα factor by the ubiquitin proteasome system. In fact the IκBα factor is a protein that sequesters NF-κB factors in the cytoplasm of non-stimulated cells (Hay et al., 1999).

Exogenous inflammatory stimuli such as viral or bacterial infection activate a signalling pathway leading to the phosphorylation of the IκBα factor. This phosphorylation occurs specifically at the Serine residues in positions 32 and 36 of the IκBα amino acid sequence. The IκBα factor is phosphorylated by the protein kinase complex Iκκ. When it is phosphorylated in this way, the IκBα factor is recognised by ubiquitin ligase SCF^{β}-TrCp (Kroll et al, 1999; Winston et al, 1999). Recognition of the IκBα factor by ubiquitin ligase SCF^{β-TrCp} leads to polyubiquitination of this factor. After ubiquitination, the IκBα factor is recognised and degraded by the proteasome. The destruction of the IκBα factor causes release of the cytoplasmic NF-κB factor. The NF-κB factor is translocated from the cytoplasm to the nucleus. Once localised in the nucleus of stimulated cells, the NF-κB factor specifically recognises the promoters of target genes and strongly activates their transcription: the inflammatory response is in place (Ben Neriah, 2002).

Numerous experimental data appear to confirm that the release of the NF-κB factor, caused by degradation of phosphorylated factor IκBα, is an essential step for inflammation to start and also for a situation of chronic inflammation to take hold (Magnani et al, 2000; Lewis and Manning, 1999).

New state-of-the-art anti-inflammatory compounds for treating acute inflammation and chronic inflammation are needed. In particular, a need exists for anti-inflammatory compounds that are both more effective and more specific than known anti-inflammatory compounds. Such anti-inflammatory compounds, because of their specificity against a biological target, would be likely to have reduced undesirable side effects, and may even have no undesirable side effects at all.

There also exists a need to develop a method for identifying compounds of therapeutic interest, more specifically anti-inflammatory compounds with increased benefit, such as those herein.

### DESCRIPTION OF THE INVENTION

### General Description of the Screening Method of the Invention

According to the invention, a method of screening potential therapeutic agents has been developed, which are selected for their specificity of action on ubiquitination of human IκBα protein by an ubiquitin ligase complex containing human β-TrCP protein.

The applicant has shown that, surprisingly, it is possible to mimic, in yeast cells, the degradation process of IκBα factor by the proteasome, a process which occurs naturally in human cells.

Surprisingly, it has been shown according to the invention that it is possible to create artificially, in yeast cells, a protein complex that has ubiquitin ligase activity and specifically recognises the SCF^{β-TrCp} complex which is produced naturally in human cells. So according to the invention, we have constructed in yeast cells, an artificial ubiquitin ligase complex containing yeast proteins associated with human β-TrCp protein. In particular, we have shown that human β-TrCP protein, when it is artificially expressed in yeast cells, binds to the yeast Skp1 protein, and said yeast Skp1 protein is contained in a yeast ubiquitin ligase protein complex. Thus, in a yeast cell into which an expression cassette coding for human β-TrCP protein has been inserted, the β-TrCP protein binds to the yeast SCF protein complex which contains (i) a catalytic core comprised of associated Skp1, Cdc53 and Hrt1 proteins, and said catalytic core is itself associated with the enzyme E2 Cdc34. It has been shown that the hybrid yeast/human protein complex is able to mimic, in yeast cells, the ubiquitin ligase activity exercised in human cells by the natural human SCF^{β-TrCp} complex.

Just as surprisingly, it has been shown according to the invention that, in yeast cells, this artificial protein complex that has the ubiquitin ligase activity of the human SCF^{β-TrCp} complex is biologically active only when this artificial complex is located in the cell nucleus. On the contrary, in human cells, the natural SCF^{β-TrCp} complex is biologically active in the cytoplasm of human cells, and inside this cell compartment it carries out the ubiquitination of a second protein that is also located in the cytoplasm, the IκBα factor. It has also been shown according to the invention that the artificial ubiquitin ligase complex which has been developed is active, in the degradation process of the IκBα factor, only when the IκBα factor is co-located in the nucleus with the said artificial ubiquitin ligase complex.

So according to the invention, we have shown that, in yeast cells, the artificial ubiquitin ligase protein complex containing human β-TrCp protein is able to carry out the ubiquitination of human IκBα factor, when the β-TrCP protein and the IκBα factor are artificially expressed in the cell nucleus.

Finally, it has also been shown that, in the yeast cells, the ubiquitination of the IκBα factor by the new artificial ubiquitin ligase complex, even though this ubiquitination is carried out in the cell nucleus and not in the cell cytoplasm, still causes degradation of the ubiquitinated IκBα factor by the proteasome.

All of these surprising results above have enabled the applicant to develop a method of screening agents able to modulate the degradation of the IκBα factor, in yeast cells, in the presence of an artificial ubiquitin ligase complex that mimics the biological activity of the natural human SCF^{β-TrCp} ubiquitin ligase complex.

The object of the invention is a method for in vitro screening of agents modulating the ubiquitination of the IκBα protein by a functional ubiquitin ligase protein complex containing the β-TrCP protein, said method comprising the following steps
(a) bringing into contact a candidate agent to be tested with recombinant yeast cells that express in their nucleus:
(i) a fusion protein containing the polypeptide IκBα and at least one first detectable protein; and
(i) a protein containing the polypeptide β-TrCP;
(b) quantifying said first detectable protein in the yeast cells, at the end of at least one predetermined period of time after bringing the candidate agent into contact with said cells;
(c) comparing the result obtained in step (b) with a control result obtained when step (a) is carried out in the absence of the candidate agent.

The aforementioned method allows those skilled in the art to determine whether an agent to be tested is able to modify the speed of degradation, or the degree of degradation, of the IκBα factor by the proteasome, in the yeast cells expressing both the β-TrCP protein and the human IκBα factor.

The aforementioned in vitro screening method, because it uses an artificial humanised ubiquitination system in yeast cells, makes it possible to screen agents that act specifically on the activity of only the human proteins expressed in these cells.

Moreover, thanks to the above method, a physiological test of screening agents active on the ubiquitin ligase system has been developed, by creating in yeast cells a metabolic pathway for protein degradation that mimics proteasome degradation of the human IκBα factor. Thus, as far as the targeted metabolic pathway of protein degradation is concerned, the invention method uses physiological conditions that are very close to the physiological conditions of protein degradation by the human proteasome.

Using the aforementioned method, it is possible to identify agents able to inhibit the speed or degree of degradation of the IκBα factor by the yeast cell proteasome. Inhibitory agents of this type, identified using the invention method, because they also inhibit degradation of the IκBα factor in human cells, are potential therapeutic agents able to inhibit or block the translocation of the NF-κB factor in the cell nucleus, and hence, to inhibit or block activation, by NF-κB, of different genes involved in inflammation, autoimmune pathologies or cancers.

Thus, the aforementioned in vitro screening method may include a subsequent step (d) consisting of positively selecting the candidate inhibitory agents for which the quantity of detectable protein measured in step (b) is lower than the comparable control value.

The invention method also makes it possible to identify agents able to increase the speed or degree of degradation of the IκBα factor by the yeast cell proteasome. Activating agents of this type are able to induce or increase the translocation of the NF-κB factor in the cell nucleus, and hence to induce or increase the activation, by NF-κB, of different genes involved in inflammation, autoimmune pathologies or cancers. Thus, according to this second aspect, the in vitro screening method of the invention makes it possible to screen proinflammatory agents. Some proinflammatory agents selected according to the method are likely to reveal therapeutic properties when they are used in low dosages or when they are administered over a short period of time, for example as agents to induce an early immune response, such as inducing a non-specific resistance reaction to the infection, or such as activating antigen presenting cells, for initiating a specific immune response to an antigen, whether by humoral mediation or cell mediation. Certain other proinflammatory agents selected according to the in vitro screening method of the invention may contain known active principles, including active principles of drugs, for which an adverse proinflammatory effect has been identified, and for which particular precautions for use in human health must be observed.

Thus, according to a further aspect, the screening method according to the invention may include a subsequent step (d) consisting of positively selecting the candidate activator agents for which the quantity of detectable protein measured in step (b) is higher than the comparable control value.

Thus an agent which “modulates” the ubiquitination of the β-TrCP protein consists (i) of an agent that increases, or, on the contrary, consists (ii) of an agent which inhibits or blocks, the degradation of the β-TrCP protein which is detected at step (b) of the screening method of the invention, with respect to control degradation of this same protein, when the method is carried out in the absence of the agent being tested.

As will have been understood, the agent modulating the ubiquitination of the β-TrCP protein can be of any kind. Said agent can be any organic or inorganic compound, and can be either a naturally occurring agent, or an agent produced, at least in part, by chemical or biological synthesis. Said agent can be a peptide or a protein, among other things. Said agent also includes any molecule already known to have a biological effect, and particularly a therapeutic effect, or on the contrary a proven or suspected toxic effect on the human body.

In the method according to the invention, once the IκBα-detectable protein fusion protein is ubiquitinated by the artificial SCF complex containing the β-TrCP polypeptide, said fusion protein undergoes proteolysis which is brought about by the multicatalyst proteasome complex. By measuring the detectable protein contained in the yeast cell at a given moment, it is possible to determine the degree of degradation of said IκBα-detectable protein fusion protein, at that given moment.

According to the invention, it has been shown that the sensitivity of the screening method described above is increased when, before putting the yeast cells into contact with the agent to be tested, the accumulation of the target fusion protein IκBα-detectable protein in the cell nucleus is enhanced.

Thus, according to a first preferred embodiment of the above method, the step (a) itself comprises the following steps:
(a1) cultivating yeast cells which express in their nucleus a fusion protein containing the polypeptide IκBα and at least one first detectable protein;
(a2) stopping the expression of said fusion protein containing the polypeptide IκBα and at least one first protein detectable by the yeast cells;
(a3) bringing the yeast cells obtained at the end of step (a2) into contact with the candidate agent to be tested.

Those skilled in the art will easily be able to stop the expression of the IκBα-detectable protein fusion protein, at a moment of their choosing, by using, to transform the yeast cells, an expression cassette in which the polynucleotide coding said fusion protein is placed under the control of a functional promoter in the yeast cells, the activation, or, on the contrary, the repression of which, is brought about by an induction agent. Those skilled in the art are familiar with many active inducible promoters in yeast cells, and some of them are described below in the description, and also in the examples.

Accumulation of the IκBα-detectable protein fusion protein in the yeast cell nuclei, in step (a1) of the method, makes it possible to obtain a strong detection signal from the detectable protein, at the start of the method. These strong signal conditions make it possible to measure the detectable protein very accurately throughout the whole method, as and when the IκBα-detectable protein fusion protein is broken down by the proteasome, after it has been ubiquitinated by the artificial SCF complex containing the β-TrCP protein. Obviously the stronger the detectable signal at the outset, the greater the sensitivity of the measurements when the method is implemented.

According to a first aspect of the above embodiment, the yeast cells express the protein containing the polypeptide β-TrCP throughout all the steps (a1), (a2) and (a3).

According to a second aspect of the above embodiment, the yeast cells express the protein containing the polypeptide β-TrCP throughout the steps (a2) and (a3) and do not express the protein containing the polypeptide β-TrCP during step (a1).

According to this second aspect, it is easy to control the expression of the protein containing the β-TrCP polypeptide by using, to transform the yeast cells, an expression cassette in which the polynucleotide coding for the protein containing the β-TrCP polypeptide is placed under the control of a functional promoter in the yeast cells, the activation, or, on the contrary, the repression of which, is brought about by an induction agent. Those skilled in the art are familiar with many active inducible promoters in yeast cells, and some of them are described below in the description, and also in the examples. Most preferably, the inducible promoter included in the expression cassette coding for the protein containing the β-TrCP polypeptide is distinct from the inducible promoter included in the expression cassette coding for the IκBα-detectable protein fusion protein. According to this preferred embodiment, a separate control is carried out respectively of (i) the expression of the IκBα-detectable protein fusion protein and (ii) the expression of the protein containing the β-TrCP polypeptide.

According to this second aspect, the IκBα-detectable protein fusion protein accumulates in the yeast cell nuclei in step (a1), in the absence of β-TrCP polypeptide. Then, in step (a2) the IκBα-detectable protein fusion protein that is no longer produced is put in the presence, in the cell nucleus, of the artificial SCF complex which contains the β-TrCP protein, the expression of which was induced. In this embodiment of the method, first the target fusion protein containing IκBα accumulates, then the effector protein for ubiquitination is expressed, that is to say the protein which contains the β-TrCP polypeptide, which initiates the degradation of the fusion protein IκBα-detectable protein. And the IκBα-detectable protein fusion protein degradation process, which can be altered by the agent under test, is measured at step (b) of the invention screening method.

According to a third aspect of the preferred embodiment of the screening method of the invention, the yeast cells express the protein containing the polypeptide β-TrCP throughout steps (a2) and (a3), and
(i) do not express the protein containing the β-TrCP polypeptide for a predetermined time, at the start of step (a1);
(ii) do express the protein containing the β-TrCP polypeptide for the remainder of step (a1).

Likewise, according to this third aspect, the fusion protein IκBα-detectable protein is expressed throughout the whole of step (a1) of the method, and expression of said fusion protein is stopped at step (a2) of the method.

According to this third aspect, expression of the protein containing the β-TrCP polypeptide is activated at a chosen time during step (a1). In these conditions, during part (ii) of step (a1), the fusion protein IκBα-detectable protein and the protein containing the β-TrCP polypeptide are simultaneously expressed in the yeast cells.

According to this third aspect, the fusion protein IκBα-detectable protein accumulates in large quantities in the yeast cell nuclei during the whole of step (a1), and the effector protein containing the β-TrCP polypeptide is expressed early in the course of step (a1), and continues to accumulate throughout steps (a2) and (a3) during which the target fusion protein is no longer synthesised. In these conditions, because of the large quantity of effector protein containing the β-TrCP polypeptide accumulated in the yeast cell nuclei, a high level of ubiquitination of the target fusion protein and, therefore, also a high level of target protein degradation by the proteasome, is promoted, which considerably increases the sensitivity of the screening method, when testing potential candidate agents inhibitory to IκBα polypeptide ubiquitination.

Preferably, according to this third aspect of the invention method, during step (a1), the expression of the IκBα-detectable protein fusion protein is activated for period T1 comprised between 0.25 hours and 10 hours, more preferably between 0.5 hours and 6 hours and most preferably between 1 hour and 4 hours.

Then, at a predetermined time t2, during the period T1, expression of the effector protein containing the β-TrCP polypeptide, is activated. Preferably, the time t2 is between [T1-8 hours] and [T1-0.1 hours], more preferably between [T1-5 hours] and [T1-0.25 hours], and most preferably between [T1-3 hours] and [T1-0.5 hours], the time t2 being, by definition, selected between the limits of the previously selected period T1.

Then, at the end of the period T1, that is after the start of step (a2), the expression of the IκBα-detectable protein fusion protein is stopped. From this moment on, only expression of the effector protein containing the β-TrCP polypeptide is activated in the yeast cells, and this activity is maintained throughout the rest of the screening procedure, that is, until the end of the procedure.

### Description of the Preferred Embodiments of the Screening Method

The preferred embodiments of the screening method of the invention are described below, particularly relating to the description of the structural and functional aspects of the various resources for implementing said method.

In general, the detectable protein which is contained in the IκBα-detectable protein fusion protein may be of any kind, such that its presence can be specifically detected in yeast cells before its proteolysis, and that the presence of proteolysed forms of detectable protein, particularly peptide fragments produced by proteolysis of said detectable protein, are not detected by the chosen method of specific detection.

As is easily understood, the ubiquitin ligase activity of the artificial protein complex containing the β-TrCP protein is followed, according to the invention method, by measuring its effect on the stability of the IκBα-detectable protein fusion protein. Addition of polyubiquitin chains to the IκBα factor by the artificial human/yeast SCF complex, leads to recognition of the ubiquitinated I(Bα factor by the proteasome, and its rapid degradation by the latter. Thanks to the expression, in yeast cells, of the factor IκBα in the form of a fusion protein, degradation of the fusion protein containing IκBα can be followed in real time by detecting the non-proteolysed detectable protein. Depending on the type of detectable protein fused to IκBα, the degradation of the fusion protein can itself be followed by known techniques, including techniques using measurement of fluorescence with a flow cytometer, a microplate reader, a fluorimeter, or a fluorescence microscope and also by colorimetric, enzymatic or immunological techniques. As an illustration, the detectable protein can be chosen from an antigen, a fluorescent protein or a protein having enzymatic activity.

When the detectable protein is an antigen it can be any type of antigen, so long as the specific antibodies for this antigen are readily available or, alternatively, can be prepared according to any method for preparing antibodies, including polyclonal or monoclonal antibodies, well known to those skilled in the art. Preferably, in this case, the detectable protein is a small sized antigen, which is not likely to interfere with recognition of the IκBα factor by the β-TrCP polypeptide. So, preferably, a peptide chain of 7 to 100 amino acids in length, more preferably 7 to 50 amino acids long, or better still, 7 to 30 amino acids long, for example 10 amino acids long, is used as the antigen. As illustration, the HA antigen with the sequence [NH₂-YPYDVPDYA—COOH] SEQ ID N^{o} 17, or a FLAG antigen with the sequence [NH₂-DYKDDDDK—COOH] SEQ ID N^{o} 18 (FLAG monomer) or with the sequence [NH2-MDYKDHDGDYKDHDIDYKDDDDK—COOH] SEQ ID N^{o} 19 (FLAG trimer) can be used. In this case, to quantify the detectable protein at step (b) of the procedure, an antibody specific to the antigen contained in the fusion protein is used, this antibody being directly or indirectly labelled. Then the quantification is done by measuring the detectable signal from the complexes formed in the yeast cells between the labelled antibody and the IκBα-antigen fusion protein. So, at step (b), when the first detectable protein is an antigen, said first detectable protein is quantified by detecting the complexes formed between said protein and the antibodies which recognise it.

When the detectable protein is an intrinsically fluorescent protein, it is, for instance, one selected from the GFP protein or one of its derivatives, the YFP protein or one of its derivatives, and the dsRED protein. For instance, among the proteins derived from the GFP protein, one of the proteins known by the names GFPMut3, Venus, Sapphire etc. can be used. An illustrative list of the GFP proteins suitable for use in the invention method is given in Table 3 at the end of the current description.

Also, the intrinsically fluorescent protein can be chosen from among autofluorescent proteins that come from various organisms, other than Aequorea Victoria. For instance, the intrinsically fluorescent protein can be chosen from the following proteins:
the CopGFP protein from Pontellina plumata, and described by D. A. Shagin et al.(2004, Mol. Biol. Evol. 21:841-850);
the TurboGFP protein, a variant of CopGFP; and described by D. A. Shagin et al., 2004 (Mol. Biol. Evol. 21:841-850);
the J-Red protein from Anthomedusae; and described by D. A. Shagin et al., 2004 (Mol. Biol. Evol. 21:841-850);
the PhiYFP protein from Phialidium sp.; and described by D. A. Shagin et al.(2004, Mol. Biol. Evol. 21:841-850);
the mAG protein, also called “monomeric Azami-Green”, from the coral Galaxeidae; and described by S. Karasawa et al. (2003, J. Biol. Chem. 278:34167-34171);
the AcGFP protein from Aequorea coerulescens, as well as its variants, and described by N. G. Gurskaya, (2003, Biochem. J. 373:403-408); and
the DsRed protein from Discosoma sp.; and described by M. V. Matz et al (1999, Nature Biotech. 17:969-973).

When the detectable protein is an intrinsically fluorescent protein, the detectable protein is quantified at step (b) of the method by measuring the fluorescent signal emitted by the IκBα-fluorescent protein fusion protein using any appropriate device. So, at step (b), when the first detectable protein is a fluorescent protein, said detectable protein is quantified by measuring the fluorescent signal emitted by said protein.

When the detectable protein is a protein with enzymatic activity, said detectable protein is chosen, for instance, from luciferase and β-lactamase. In this case, the detectable protein is quantified at step (b) of the method by measuring the amount of the compound or compounds produced by enzymatic conversion of the substrate. When the product of enzymatic activity is coloured, the measurement can be done by colorimetry. When the product of enzymatic activity is fluorescent, the intensity of the fluorescent signal emitted by said product is measured using any suitable device for measuring fluorescence. So, at step (b), when the first detectable protein is a protein having enzymatic activity, said detectable protein is quantified by measuring the quantity of substrate transformed by said protein.

In a specific embodiment of the screening method according to the invention, the protein containing the β-TrCP polypeptide also consists of a fusion protein containing, in addition to the β-TrCP polypeptide, a detectable protein also. In this specific embodiment, the level of β-TrCP polypeptide expression in yeast cells, over time, can be followed by detecting and, optionally, by quantifying the detectable protein contained in the protein containing the β-TrCP polypeptide. This specific embodiment is mainly used when positively or negatively controlling expression of the protein recognising the β-TrCP polypeptide, at different sub-steps of step (a) of the method. The detectable protein in the polypeptide containing the β-TrCP polypeptide is chosen from an antigen, a fluorescent protein and a protein having enzymatic activity. Preferably, the detectable protein in the protein containing the β-TrCP polypeptide is different from the detectable protein in the IκBα-detectable protein fusion protein, allowing expression of the factor IκBα and expression of the β-TrCP polypeptide in yeast cells, to be followed independently.

As already mentioned in this description, degradation of the human IκBα target polypeptide by the yeast cell proteasome occurs only when the IκBα-detectable protein fusion protein and the protein containing the human β-TrCP polypeptide are both located in the yeast cell nuclei.

In particular, the applicant has shown, as is illustrated in the examples, that factor IκBα is phosphorylated at serine residue 32 only in the nucleus of yeast cells, and that it does not undergo phosphorylation in the cytoplasm. A posteriori, the phosphorylation of the serine residue at position 32 of factor IκBα, in yeast cells, at least partly explains the reason why, in yeast cells, ubiquitination of this factor can only occur in the cell nucleus.

Hence, in order to carry out the screening method of the invention, all means must be in place for allowing simultaneous nuclear localisation of the fusion protein IκBα-detectable protein and the protein recognising the β-TrCP polypeptide.

Preferably the fusion protein IκBα-detectable protein and the protein containing the β-TrCP polypeptide both contain a peptide allowing both these proteins to localise in the nucleus of yeast cells.

So, preferably, the fusion protein IκBα-detectable protein and the protein containing the β-TrCP polypeptide both contain in their amino acid sequence at least one nuclear localisation peptide (“NLS”) which is functional in eucaryotic cells, and more especially in yeast cells. Each of the proteins contains, independently of the other, 1, 2, 3 or 4 nuclear localisation peptides. According to another aspect, each of these proteins contains, independently of the other, 1 to 4 copies of a nuclear localisation peptide.

Preferably, the nuclear localisation peptide or peptides are selected from the following peptides:
the NLS peptide derived from the big antigen of the SV40 virus having the amino acid sequence SEQ ID N^{o}24;
the nucleoplasmin NLS peptide having the amino acid sequence SEQ ID N^{o}20;
an NLS peptide of the yeast alpha 2 repressor selected from sequences SEQ ID N^{o} 21 and SEQ ID N^{o} 22;
an NLS peptide of the yeast Gal4 protein having the amino acid sequence SEQ ID N^{o}23.

In the examples, the fusion protein IκBα-detectable protein and the protein containing the β-TrCP polypeptide both contain the nuclear localisation peptide with sequence SEQ ID N^{o}24.

Preferably, the IκBα-detectable protein fusion polypeptide consists of an amino acid chain containing, from the NH₂ terminus to the COOH terminus respectively, (i) the sequence of the detectable protein, (ii) the nuclear localisation sequence NLS and (iii) the IκBα sequence.

Firstly, in the fusion polypeptide, the GFP sequence and the NLS sequence can be directly bonded to each other by a peptide bond. Similarly, the NLS sequence and the IκBα sequence can be directly bonded to each other by a peptide bond.

According to another aspect, the GFP sequence and the NLS sequence can be separated, in the fusion polypeptide sequence, by a first spacer peptide.

According to yet another aspect, the NLS sequence and the IκBα sequence can be separated, in the fusion polypeptide sequence, by a second spacer peptide. Advantageously, the spacer peptide(s), when present, range in size from 1 to 30 amino acids, preferably from 1 to 15 amino acids and most preferably from 2 to 10 amino acids long.

According to a preferred embodiment, the protein containing the IκBα polypeptide consists of the protein with the amino acid sequence SEQ ID N^{o}2, which can be coded for by the nucleic acid sequence SEQ ID N^{o}1. The protein with sequence SEQ ID N^{o}2 consists of, from the NH₂ terminus to the COOH terminus respectively, (i) the detectable protein sequence GFP(yEGFP3) running from the amino acid position 1 to amino acid position 240, (ii) a first spacer peptide running from amino acid position 241 to amino acid position 243, (iii) the SV40 virus big-T antigen NLS peptide running from amino acid position 244 to amino acid position 250, (iv) a second spacer peptide running from amino acid position 251 to amino acid position 255 and (v) the IKBc polypeptide running from amino acid position 256 to amino acid position 572. The nucleic acid of sequence SEQ ID N^{o}1 consists of, from the 5′ end to the 3′ end respectively, (i) the sequence coding for the detectable protein GFP(yEGFP3) running from nucleotide position 1 to nucleotide position 714, (ii) the sequence coding for the first spacer peptide running from nucleotide position 715 to nucleotide position 729, (iii) the sequence coding for the SV40 virus big-T antigen NLS peptide running from nucleotide 730 to nucleotide 750, (iv) the sequence coding for the second spacer peptide running from position 751 to nucleotide 765 and (v) the sequence coding for the IκBα polypeptide running from nucleotide 766 to nucleotide 1719.

Preferably, the protein containing the βTrCP polypeptide consists of an amino acid chain that contains, from the NH₂ terminus to the COOH terminus respectively (i) the sequence of a second detectable protein, (ii) the nuclear localisation sequence, NLS, and (iii) the βTrCP sequence.

According to a preferred embodiment, the protein containing the β-TrCP polypeptide consists of the protein with the amino acid sequence SEQ ID 4, which is coded for by the nucleic acid sequence SEQ ID N^{o}3. The protein with sequence SEQ ID N^{o}4 consists of, from the NH₂ terminus to the COOH terminus respectively, (i) the detectable protein sequence GFP(yEGFP3) running from the amino acid position 1 to amino acid position 240, (ii) a first spacer peptide running from amino acid position 241 to amino acid position 243, (iii) the SV40 virus big-T antigen NLS peptide running from amino acid position 244 to amino acid position 250, (iv) a second spacer peptide running from amino acid position 251 to amino acid position 255 and (v) the β-TrCP polypeptide running from amino acid position 256 to amino acid position 860. The nucleic acid of sequence SEQ ID N^{o}3 consists of, from the 5′ end to the 3′ end respectively, (i) the sequence coding for the detectable protein GFP(yEGFP3) running from nucleotide position 1 to nucleotide position 714, (ii) the sequence coding for the first spacer peptide running from nucleotide position 715 to nucleotide position 729, (iii) the sequence coding for the SV40 virus big-T antigen NLS peptide running from nucleotide 730 to nucleotide 750, (iv) the sequence coding for the second spacer peptide running from position 751 to nucleotide 765 and (v) the sequence coding for the β-TrCP polypeptide running from nucleotide 766 to nucleotide 2538.

According to yet another aspect, the screening method according to the invention is characterised in that the recombinant yeast cells are transformed with:

(1) a first polynucleotide that contains (a) an open reading frame coding for (i) the fusion protein containing the IκBα polypeptide, (ii) a nuclear localisation sequence and (iii) a first detectable protein, and (b) a functional regulatory sequence which in yeast cells leads to expression of said open reading frame; and (2) a second polynucleotide that contains (a) an open reading frame coding for (i) the protein containing the β-TrCP polypeptide, ii) a nuclear localisation sequence and (iii) a functional regulatory sequence which in yeast cells leads to expression of said open reading frame;

The above polynucleotide (1) can consist of the nucleic acid of sequence SEQ ID N^{o}1.

The above polynucleotide (2) can consist of the nucleic acid of sequence SEQ ID N^{O}3.

### Preferred Nucleic Acids, Expression Vectors and Transformed Yeast Cells According to the Invention.

According to the invention nucleic acids are synthesised, so that, when they are introduced into yeast cells, they cause respectively expression of the fusion protein IκBα-detectable protein and the protein containing the β-TrCP polypeptide in these cells, and more particularly in the nuclei of yeast cells.

Firstly, each of the nucleic acids synthesised contains a coding sequence, also called “open reading frame” or “ORF”, that codes for the protein of interest, being respectively the fusion protein IκBα-detectable protein, or the protein containing the β-TrCP polypeptide, said protein of interest also containing in its sequence at least the sequence of a nuclear localisation peptide. Some illustrative examples of nucleic acids according to the invention are the nucleic acids of sequence SEQ ID N^{o}1 and SEQ ID N^{o}3, the structures of which have been described previously in the description.

Each of the nucleic acids also contains a regulatory sequence containing a promoter functional in yeast cells.

According to a first preferred embodiment, the promoter functional in yeast cells consists of a constitutive promoter that can be chosen from the promoters PGK1, ADH1, TDH3, LEU2 and TEF1.

Preferably, with the aim of precisely controlling the periods during which the IκBα-detectable protein fusion protein and the protein containing the β-TrCP polypeptide respectively are expressed, each of the nucleic acids contains, as a promoter, a promoter called “inducible”, that is to say a promoter functional in yeast cells that is sensitive to the action of an inducing agent. It is possible to use a promoter which, when the inducing agent is added to the yeast cell culture medium, activates expression of the sequence coding for the protein of interest, which is under its control. It is also possible to use a promoter which, when the inducing agent is added to the yeast cell culture medium, suppresses or blocks expression of the sequence coding for the protein of interest, which is under its control.

Thus, according to a second preferred embodiment of a promoter, the inducible promoter contained in the nucleic acids of the invention is chosen from CUP1, GAL1, MET3, MET25, MET28, SAM4 and PHO5.

In a preferred embodiment, the nucleic acid or polynucleotide coding for the fusion protein IκBα-detectable protein contains the GAL1 regulatory sequence which, in the presence of glucose, activates expression of the open reading frame coding for the fusion protein containing the IκBα polypeptide.

So, in an advantageous embodiment of the screening method of the invention, the expression of the fusion protein containing the IκBα factor occurs in a transitory fashion during the screening. After having been induced for a fixed time varying from 20 minutes to 24 hours, expression of the protein containing IκBα is selectively stopped (by a procedure known to those skilled in the art as “promoter shut off”) before exposing the cells to the molecules to be screened. This stopping of expression is achieved by the addition to (or removal from) the culture medium of a molecule able to suppress activity of the promoter controlling expression of the tripartite protein containing IκBα.

Thus, when the IκBα-detectable protein fusion protein is expressed under the control of the GAL1 gene promoter, expression of this promoter is then suppressed by adding glucose, to a final concentration of 2%, to the culture medium. Stopping de novo synthesis of the fusion protein containing IκBα allows real-time measurement of its stability, by determining, for example, the fluorescence of the yeast cells over time after stopping synthesis, in the embodiment in which said fusion protein contains a protein detectable by intrinsic fluorescence, such as GFP or a protein derived from GFP.

In another particularly advantageous embodiment of the screening method according to the invention, the transient expression of the fusion protein containing the IκBα factor is associated with the equally transient expression of the protein containing the β-TrCP polypeptide. In this embodiment, the fusion protein containing the IκBα polypeptide is expressed during the chosen time period T1, for example by using yeast cells that express the fusion protein containing the IκBα polypeptide under the control of the GAL1 promoter, and which are grown in the presence of 0.5 to 4% galactose for the duration of T1. At the point of t2, expression of the protein containing the β-TrCP polypeptide is induced. This induction is achieved, in cells expressing the protein containing β-TrCP under the control of the CUP1 gene promoter for example, by adding copper sulphate at a concentration comprised between 0.05 mM and 5 mM to the culture medium. At the end of the time period T1, expression of the fusion protein containing IκBα is stopped by adding glucose to a concentration comprised between 0.5 and 2% to the culture medium. This addition of glucose has no effect on the expression of the protein containing β-TrCP under the gene promoter CUP1. Thus, in this embodiment of the method, accumulation of ubiquitin ligase containing β-TrCP continues while the de novo synthesis of the fusion protein containing IκBα stops.

Thus, in a specific embodiment of the screening method according to the invention, the nucleic acid or polynucleotide coding for the protein containing the β-TrCP polypeptide contains the regulatory sequence CUP1, which activates, in the presence of copper sulphate, expression of the open reading frame coding for a protein containing the β-TrCP polypeptide.

Thus, a further object of the invention is an expression cassette functional in yeast cells containing a coding polynucleotide including an open reading frame encoding the fusion protein which contains the polypeptide, the IκBα polypeptide and at least one first detectable protein, and a regulatory sequence functional in yeast cells that causes expression of said open reading frame.

Such an expression cassette can consist of the nucleic acid, sequence SEQ ID N^{o}1 according to the invention, that codes for the fusion protein GFP-NLS-IκBα, sequence SEQ ID N^{o}2.

The invention also concerns a expression cassette functional in yeast cells including a polynucleotide which contains an open reading frame encoding a protein containing the β-TrCP polypeptide and a regulatory sequence functional in yeast cells which leads to expression of said open reading frame.

Such an expression cassette can consist of the nucleic acid, sequence SEQ ID N^{o}3 according to the invention, that codes for the fusion protein GFP-NLS-βTrCP, sequence SEQ ID N^{o}4.

According to a first preferred embodiment of such an expression cassette, the regulatory sequence contains an inducible promoter functional in yeast cells, such as a promoter chosen from the promoters PGK1, ADH1, TDH3, LEU2 and TEF1.

According to a second preferred embodiment of such an expression cassette, in one or other of the above expression cassettes, or in both, the regulatory sequence contained in said polynucleotide, the regulatory sequence contained in the second polynucleotide, or both regulatory sequences contain a promoter functional in yeast cells sensitive to the action of an inducing agent, which is also called an inducible promoter.

Most preferably, the inducible promoter functional in yeast cells is chosen from CUP1, GAL1, MET3, MET25, MET28, SAM4 and PHO5.

Thus, in another advantageous embodiment of the screening method according to the invention, the yeast cells are transformed by (i) a nucleic acid or polynucleotide containing the sequence coding for the fusion protein IκBα-detectable protein along with (ii) the nucleic acid or polynucleotide coding for the protein containing the β-TrCP polypeptide, which is present in a non-integrated form, for example in the form of vectors functional in the yeast cells and which carry at least one origin of replication functional in yeast cells.

In yet another embodiment of the screening method according to the invention, the recombinant yeast cells have, in a form integrated into their genome, the nucleic acid or polynucleotide containing the sequence coding for the fusion protein IκBα-detectable protein as well as the nucleic acid or polynucleotide coding for the protein containing the β-TrCP polypeptide, as illustrated in the examples.

In general, to use the screening method of the invention, it is advantageous to use yeast cells with a highly permeable membrane, specifically, good permeability to the agents to be tested by the method.

To use the preferred embodiment of the screening method of the invention, in which expression of the two proteins of interest is under the control of inducible promoters, it is also advantageous to use yeast cells with a highly permeable membrane for the inducer substances to which said inducible promoters are sensitive.

Thus, in another preferred embodiment of the screening method of the invention, yeast strains are used which have a genome containing one or several mutations which increase permeability to the substances under test, such as mutations inactivating the PDR1 and PDR3 genes, two genes which code for transcription factors that, in yeast, control expression of plasma membrane transporters (Vidal et al, 1999, Nourani et al, 1997).

Preferably, yeast strains are used which have the genetic background of the Saccharomyces cerevisiae yeast strain W303 described by Bailis et al. (1990), or any another characterised strain of said yeast Saccharomyces cerevisiae.

Transformation of yeast cells by exogenous DNA is, preferably, carried out using techniques known to those skilled in the art, specifically the technique described by Schiestl et al. (1989). The construction of different yeast strains was done using known genetic techniques (growth, sporulation, dissection of the asci and phenotypic analysis of the spores) described particularly by Sherman et al. (1979) and reverse genetic techniques described particularly by Rothstein (1991).

In accordance with the invention, yeasts are transformed, preferably, with plasmids constructed according to classic molecular biology techniques, particularly according to the protocols described by Sambrook et al. (1989) and Ausubel et al. (1990-2004).

Thus, another object of the invention consists of an expression vector characterised in that it contains an expression cassette such as defined in the current description.

A first vector conforming to the invention is the vector pCSY226-NLS-IκBα which is described in the examples, and which was used in the construction of the yeast strain CYS135 deposited in the Collection Nationale de Cultures de micro-organismes at the Institut Pasteur de Paris under the accession number I-3187.

A second vector conforming to the invention is the vector pCSY226-NLS-β-TrCP which is described in the examples, and which was used in the construction of the yeast strain CYs135 deposited in the Collection Nationale de Cultures de micro-organismes at the Institut Pasteur de Paris under the accession number I-3187.

The present invention also concerns a recombinant yeast strain containing, in a form integrated into the genome,

(i) a first polynucleotide that contains an open reading frame coding for the fusion protein containing the polypeptide, the IκBα polypeptide and at least one first detectable protein, and a regulatory sequence functional in yeast cells which controls expression of said open reading frame; and

(ii) a second polynucleotide that contains an open reading frame coding for a protein containing the β-TrCP polypeptide and a regulatory sequence functional in yeast cells which controls expression of said open reading frame;

Specifically, the invention concerns a recombinant yeast strain as defined above, which consists of the yeast strain CYS135 deposited in the Collection Nationale de Cultures de microorganismes at the Institut Pasteur de Paris (CNCM) under accession number I-3187.

The invention also concerns the tools or kit for the screening of agents modulating the ubiquitination of the IkBs protein by a functional ubiquitin ligase protein complex containing the β-TrCP protein, characterised in that it contains:

(i) a first expression vector containing an expression cassette coding for the fusion protein containing the IκB═ polypeptide as defined above; and

(ii) a second expression vector containing an expression cassette coding for the protein containing the β-TrCP polypeptide as defined above.

The invention also concerns the tools or kit for the screening of agents modulating the ubiquitination of the IκBα protein by a functional ubiquitin ligase protein complex containing the β-TrCP protein, characterised in that it includes recombinant yeast cells containing, in a form integrated into their genome, respectively:

(i) an expression cassette coding for the fusion protein containing the IκBα polypeptide as defined above; and

(ii) an expression cassette coding for the protein containing the β-TrCP polypeptide as defined above.

Preferably, the above tools or kit contain recombinant yeast cells of the strain CYS135 deposited at the CNCM under accession number I-3187.

The screening method according to the invention, allows visualisation of the activity of the ubiquitin ligase SCFP^{β-TrcP} in relation to the human IκBα factor, substrate for the proteasome-ubiquitin pathway of protein degradation. The method is particularly advantageous for screening molecules or agents suitable for use in conditions related to the activation of NF-κB factors and to NF-κB pathway dysfunction in humans such as inflammatory and immune syndromes, certain cancers, some conditions such as “reperfusion injury” and fungal, bacterial and viral infections.

The main advantages of the screening method of the invention are the following:
simplicity of use: SCF^{β-TrCP} ubiquitin ligase activity relative to the IκBα factor is easily induced thanks to the controlled expression of the human IκBα and β-TrCP factors in yeast cells. Furthermore, when the IκBα factor is expressed as a hybrid protein fused with an intrinsically fluorescent protein, such as GFP, the activity of the artificial ubiquitin ligase SCF^{β-TrCP}, relative to the IκBα factor, is measured directly by quantification of the fluorescence emitted by the hybrid protein. Similarly, when the IκBα factor is expressed as a hybrid protein, fused to a protein such as luciferase, the activity of the artificial ubiquitin ligase SCF^{β-TrCP}, relative to the IκBα factor, is measured directly by quantification of the luminescence emitted by the hybrid protein in the presence of a substrate such as fluorescein.
suitability in a therapeutic context: the activity of the artificial ubiquitin ligase SCF^{β-TrCP} relative to the IκBα factor is followed according to a functional test performed on whole cells. Thus the in vitro screening method according to the invention allows selection of molecules able to activate or inhibit degradation of IκBα in a context similar to that of their eventual therapeutic use.
specificity: although it is used in vitro in cells, the screening method according to the invention is specific, because it depends on the co-expression of the two human proteins IκBα and β-TrCP in an organism heterologous with humans. Molecules selected thanks to the screening method of the invention will be specific for the pair ubiquitin ligase β-TrCP/protein substrate IκBα, and therefore will not be molecules selected, for example, because of their ability to interfere with one of the extensive range of pathways signalling inducing IκBα degradation in human cells. In fact, at the start of the screening method according to the invention, the degradation of IκBα, by the intermediate artificial ubiquitin ligase SCF^{β-TrcP}, is induced by a completely artificial and totally reproducible metabolic pathway, such as, for example, the addition of glucose to block GAL1 promoter activity when IκBα is expressed under the control of this promoter.
the stability of the recombinant yeast strains: techniques for integration at a chosen position in the yeast chromosome and for target replacement of genes allow construction of recombinant yeast strains expressing the hybrid human proteins containing either IκBα or β-TrCP from yeast chromosomes. Thus these recombinant yeast strains are genetically stable and can be multiplied and retained indefinitely.
rapidity of growth and screening: yeast is a fast growing, high yield organism. Specifically, the screening method of the invention is, for preference, performed by culturing yeast cells in a complete culture medium, in which the growth of yeast cells is particularly rapid and the yield particularly high, which allows recovery of a large quantity of recombinant yeast cells for conducting a large number screening tests simultaneously.
low cost: yeast is a microorganism for which culture, storage and characterisation are not expensive,
automation of the screening method of the invention: yeast is a microorganism that can be cultured in small volumes, at low temperature, in a standard atmosphere, in air, which makes it particularly suitable for automated screening (robotics).

The screening method according to the invention is useful specifically for selecting and characterising active agents such as anti-inflammatory, anticancer and antiviral agents and agents for use in fungal, bacterial or viral infections.

Furthermore, the present invention is illustrated, without being in any way limited, by the following figures and examples.

### FIGURES

FIG. 1 illustrates the ability of Skp1 yeast proteins and β-TrCP human proteins to interact in yeast cells.

On the x-axis: plasmids present in the transformed yeast cells;On the y-axis, β-galactosidase activity, expressed in nanomoles of substrate transformed per minute per mg of cell protein.

FIG. 2 illustrates localisation, in yeast cells, of human proteins IκBα and β-TrCP according to whether or not they are fused to an NLS sequence of SV40. Top line: fluorescence microscopy images of cell nucleus DNA stained with Hoescht 333-42 dye.

Bottom line: fluorescence microscopy images showing the localisation of GFP expression in the cells.

A: Cells transformed by GFP-NLS-β-TrCP vector; B: Cells transformed by GFP-β-TrCP vector; C: Cells transformed by GFP-NLS-IκBα vector; D: Cells transformed by GFP-IκBα vector.

FIG. 3 shows how the presence of the human IKB(X protein in the nuclei of yeast cells leads to its phosphorylation at serines 32 and 36. The figure shows a gel electrophoresis image of cell proteins of recombinant yeast strains CYS22 and CYS126, respectively.

FIG. 4 shows, by epifluorescent microscopy, the degradation of the tripartite fusion protein GFP-NLS-IκBα in the yeast cells which, at the same time, express the tripartite fusion protein Flag-NLS-β-TrCP.

FIGS. 4A to 4D show fluorescence microscopy images: upper line, cell nucleus DNA stained with Hoescht 333-42 dye; lower line, fluorescence microscopy images showing the localisation of GFP expression in the cells.

FIG. 4A: results obtained with recombinant yeast strain CYS22;FIG. 4B: results obtained with recombinant yeast strain CYS61.

FIG. 4C: results obtained with recombinant yeast strain CYS126.

FIG. 4D: results obtained with recombinant yeast strain CYS135. On the x-axis: the different times in minutes after adding glucose to the cell cultures.

FIG. 5 shows, by measurement of the fluorescence produced, the degradation of the tripartite fusion protein GFP-NLS-IκBα in the yeast cells which express or do not express the tripartite fusion protein Flag-NLS-β-TrCP.

The results are given for the recombinant yeast strains CYS135, CYS126, CYS61 and CYS22, respectively, which are labelled in boxes on the graph.

On the x-axis: the time in minutes after adding glucose to the cell cultures;On the x-axis: average intensity of the fluorescence, expressed in arbitrary units of fluorescence.

FIG. 6 shows, by Western Blot type biochemical analysis, the degradation of the tripartite fusion protein GFP-NLS-IκBα in yeast cells which, at the same time, express the tripartite fusion protein Flag-NLS-β-TrCP.

Western blotting gel images revealed with anti-GFP antibodies and FLAG anti-peptide antibodies.

On the x-axis: the time in minutes after adding glucose to the cell cultures;

The results are shown for the following recombinant yeast strains; CYS22 (FIG. 6A), CYS61 (FIG. 6B), CYS126 (FIG. 6C) and CYS135 (FIG. 6D).

FIG. 7 shows, by Western Blot type biochemical analysis, the degradation of the mutated tripartite fusion protein GFP-NLS-IκBα[S3236A] in which the phosphorylation sites Ser32 and Ser36 have been replaced by Ala residues, mutations that, in human cells, make the protein non-degradable.

Western blotting gel images revealed with anti-GFP antibodies and FLAG anti-peptide antibodies.

On the x-axis: the time in minutes after adding glucose to the cell cultures;

The results are shown for the following recombinant yeast strains; CYS138 (FIG. 7A) and CYS139 (FIG. 7B).

FIG. 8 shows, by epifluorescent microscopy analysis, the degradation of the tripartite fusion protein GFP-NLS-IκBα[S3236A] in the yeast cells which, at the same time, express the tripartite fusion protein Flag-NLS-β-TrCP.

FIGS. 8A to 8B show fluorescence microscopy images: upper line, cell nucleus DNA stained with Hoescht 333-42 dye; lower line, fluorescence microscopy images showing the localisation of GFP expression in the cells.

FIG. 8A: results obtained with recombinant yeast strain CYS138;FIG. 8B: results obtained with recombinant yeast strain CYS139.

On the y-axis: the different times in minutes after adding glucose to the cell cultures.

FIG. 9 shows, by measurement of the fluorescence emitted, the degradation of the tripartite fusion protein GFP-NLS-IκBα[S3236A] in the strains of yeast described herein.

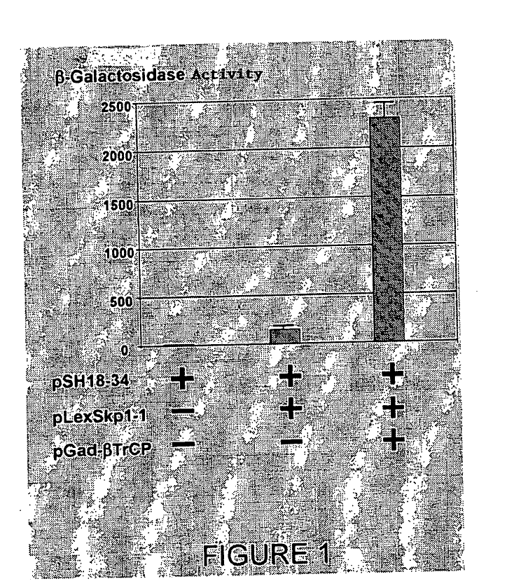

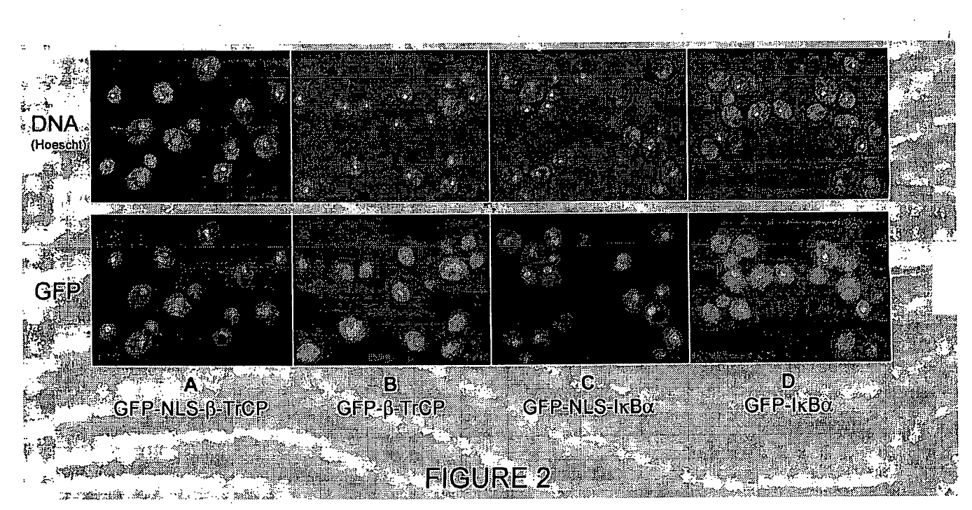

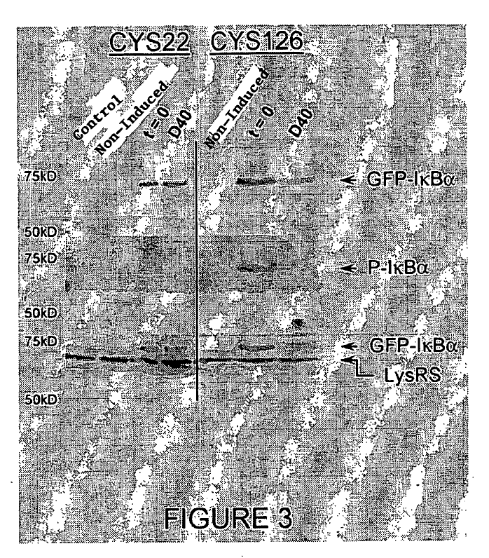

The results are given for the recombinant yeast strains CYS138 and CYS139, respectively, which are labelled in boxes on the graph.

On the x-axis: the time in minutes after adding glucose to the cell cultures;On the x-axis: average intensity of the fluorescence, expressed in arbitrary units of fluorescence.

### EXAMPLES

### Examples 1 to 3

### Construction of Recombinant Vectors According to the Invention

### A. Materials and Methods for Examples 1 to 3.

### A.1. Summary of the Polynucleotide Sequences Used

The sequence of the IKcBo protein is that described in Strausberg et al. (PNAS (1999), 99(26): 16899-16903).

The sequence of the β-TrCP receptor sub-unit of the ubiquitin ligase complex SCF^{β}-TrCP is that described in Yaron et al. (Nature (1998) 396(6711): 590-594).

The sequence of the GFP gene from Aequora Victoria, optimised for expression in yeast (yEGFP3), and its product Green Fluorescent Protein mut3, (hereafter called GFP), is that described by Cormack et al. (Gene (1996) 173 (1): 33-38).

The nuclear localisation signal “NLS” sequence of the SV40 virus big-T antigen is a translation of the nucleic acid sequence,

5′-ACCTCCAAAAAAGAAGAGAAAGGTCGAATT-3′ (SEQ ID N^{o}25).

The sequence of the pRS306 plasmid is that described by Sikorski and Hieter (Genetics (1989) 122(1): 19-27).

The sequence of the pRS304 plasmid is that described by Sikorski and Hieter (Genetics (1989) 122(1): 19-27).

The sequence of the pRS314 plasmid is that described by Sikorski and Hieter (Genetics (1989) 122(1): 19-27).

The sequence of the pRS316 plasmid is that described by Sikorski and Hieter (Genetics (1989) 122(1): 19-27).

The sequence of the plasmid pSH18-34, which contains four LexA operators upstream of the LacZ gene, is that described by Hanes et Brent (Cell (1989), 57:1275-1293)

The sequence of the pLexSkp1-1 plasmid, which expresses the Skp1 protein fused with the LexA protein, is that described in Patton et al. (Genes & Dev (1998), 12 :692-705)

The sequence of the pGADβTrCP plasmid, which expresses the human β-TrCP protein fused to the activator domain of the Gal4 yeast transcription factor is that described in Margottin et al. (Molec. Cell (1998), 1 :565-574).

The sequence of the GAL1 promoter gene from the yeast S. cerevisiae used in the following descriptions is that described by Johnston and Davis (Mol. Cell. Biol. (1984) 4 (8): 1440-1448).

The sequence of the MET3 promoter gene from the yeast S. cerevisiae used in the following descriptions is that described by Cherest et al. (Mol. Gen. Genet. (1987) 210 (2): 307-313).

The sequence of the MET28 promoter gene from the yeast S. cerevisiae used in the following descriptions is that described by Kuras et al. (EMBO J. (1996) 15(10): 2519-2529).

The sequence of the TEF1 promoter gene from the yeast S. cerevisiae used in the following descriptions is that described by Schaaff-Gerstenschlager et al. (Eur. J. Biochem. (1993) 217 (1): 487-492).

The sequence of the SAM4 promoter gene from the yeast S. cerevisiae used in the following descriptions is that described by Thomas et al. (J. Biol. Chem. (2000) 275(52): 40718-40724).

The sequence of the MET25 promoter gene from the yeast S. cerevisiae used in the following descriptions is that described by Keijan et al. (Nucleic Acids Res.(1986) 14(20): 7861-7871).

The sequence of the PHO5 promoter gene from the yeast S. cerevisiae used in the following descriptions is that described by Feldman et al. (EMBO J. (1994) 13(24): 5795-5809).

The sequence of the CUP1 promoter gene from the yeast S. cerevisiae used in the following descriptions is that described by Karin et al. (PNAS (1984) 81(2): 337-341).

The sequence of the PGK1 promoter gene from the yeast S. cerevisiae used in the following descriptions is that described by Bolle et al. (Yeast (1992) 8(3): 205-213).

The sequence of the ADH1 promoter gene from the yeast S. cerevisiae used in the following descriptions is that described by Bennetzen and Hall (J. Biol. Chem. (1982) 257(6): 3018-3025).

The sequence of the TDH3 promoter gene from the yeast S. cerevisiae used in the following descriptions is that described by Arroyo et al. Unpublished (1996), direct submission to MIPS.

The sequence of the LEU2 promoter gene from the yeast S. cerevisiae used in the following descriptions is that described by Rad et al. (Yeast (1991) 7(5): 533-538).

### A.2. Conventions Used

The descriptions use the nomenclature and typographical rules used by the Saccharomyces cerevisiae yeast biology community.
the name of the wild type gene is given in italicised upper case, for example: GAL1.
the name of the mutated form of the gene is given in italicised lower-case, the allele number, if known, follows after a hyphen; for example cup1-1.
the name of a non-functional allele in a gene is given in lower case followed by two colons followed by the name of the functional gene, e.g. ppr1::TRP1 (in this example the non-functional gene ppr1 has been interrupted by the functional gene TRP1).

Alternatively, a non-functional gene can be indicated by the “delta” symbol with the name, for example gal4Δ
the name of the protein and that of the gene coding for it is given in lower case except for the first letter, which is upper case, e.g. Gal4 (alternatively, one can use the same symbol followed by a p, for example Gal4p).

### A.3. Preliminary Comments About Construction of the Plasmids

All the plasmids were constructed using classical molecular biology techniques according to the protocols described by Sambrook et al. (in Molecular Cloning, Laboratory Manual, 2nd edition, (1989), Cold Spring Harbor, N.Y.) and Ausubel et al., (in Current Protocols in Molecular Biology, (1990-2004), John Wiley and Sons Inc, N.Y.). Cloning, replication and generation of plasmid DNA were performed in the DH10B strain of Escherichia coli.

### Example 1

### Construction of Plasmids Able to Express the Fusion Proteins GFP-IκBα and GFP-NLS-IκBα in Yeast

The following plasmids can express derivatives of the human IκBα protein fused with a variant of Green Fluorescent Protein (GFP) from Aequora Victoria, in the yeast Saccharomyces cerevisiae. Depending on the plasmid construction, the fusion proteins do or do not contain the nuclear localisation sequence from the big-T antigen of the SV40 virus. The introduction of this sequence will cause proteins that contain it to be directed to the nuclear compartment of the cell. A 620 base-pair (bp) fragment corresponding to the GAL1 gene promoter (pGAL1l) of the yeast Saccharomyces cerevisiae was amplified by Polymerase Chain Reaction (PCR) from the genomic DNA of a wild type S. cerevisiae strain, X2180-1A, using oligonucleotides “pGAL1(Asp)Forw”, sequence
5′-GCTGGGTACCTTAATAATCATATTACATGGCATTA-3′ [SEQ ID N^{o}6] and “pGAL1(EcoRI)Rev”, sequence
5′-GGCGGAATTCTATAGTTTTTTCTCCTTGACGTTA-3′ [SEQ ID N^{o}7].

The resulting fragment was digested with restriction enzymes Asp7181 and EcoRI and inserted into the S. cerevisiae-E. coli shuttle plasmid pRS306, previously digested with the enzymes Asp7181 and EcoRI, to produce the vector pRS306-pGAL1.

A 720 base-pair (bp) fragment from vector pUC19-yEGFP3, and corresponding to a variant of the gene coding for the Green Fluorescent Protein (GFP) of Aequora victoria, in which the sequence had been optimised for expression in yeast (yEGFP3), was amplified by Polymerase Chain Reaction (PCR), using the oligonucleotides “GFPEcoR15”, sequence 5′-GGTCGGAATTCATGTCTAAAGGTGAAGAATTATTC-3′ [SEQ ID N^{o}8] and “PBamHI(SmaI/SrfI PstI)3′”, sequence

5′-GGCGGGATCCGCCCGGGCTCTGCAGTTTGTACAATTCATCCATACC-3′ [SEQ ID N^{o}9]. The resulting fragment was digested with restriction enzymes BarnHI and EcoRI and inserted into plasmid pRS306-pGAL1, previously digested with the enzymes BamHI and EcoRI, to produce the vector pRS306-pGAL1-yEGFP3.

A 340 base-pair (bp) fragment corresponding to the ADH1 gene terminator signal (tADH1) of the yeast Saccharomyces cerevisiae was amplified by Polymerase Chain Reaction (PCR) from the genomic DNA of a wild type S. cerevisiae strain, X2180-1A, using oligonucleotides “TermADH1(NotIBstXI)5′”, sequence

5′-GGCGGCGGCCGCCACCGCGGTGGGCGAATTTCTTATGATTTATG-3′ [SEQ ID N^{o}10] and “TermADH1(SacI)3′”, sequence

5′-GGCGGAGCTCTGGAAGAACGATTACAACAG-3′ [SEQ ID N^{o}11].

### The resulting fragment was digested with restriction enzymes SacI and NotI and inserted into plasmid pRS306-p Gal1-yEGFP3, previously digested with the enzymes SacI and NotI, to produce the vector pCSY226.

The gene coding for the protein IκBα was purified from the plasmid pGad1318-IkBa by digestion with the restriction enzyme XbaI followed by treatment with Klenow DNA polymerase I in order to remove the overhang and give a blunt 3′ end, and then a second digestion with BamHI for the 5′ end of the gene. The fragment was cloned into plasmid pCSY226, prepared by a KpnI restriction digest, followed by treatment with Klenow fragment and then digestion with restriction enzyme BamHI. The resulting vector has been called pCSY226-IκBα.

A version of this vector also includes the nuclear location sequence NLS. This was obtained by synthesising a pair of oligonucleotides complementary to the sequences “NLS-5′”: 5′ACCTCCAAAAAAGAAGAGAAAGGTCGAATT-3′ [SEQ ID N^{o}12], and

“NLS-3′”: 5′-AATTCGACCTTTCTCTFCTCTTGGAGGT-3′ [SEQ ID N^{o}26].

and rehybridising them to form a double-stranded DNA. This DNA fragment was then incorporated into the vector pCSY226-IκBα digested with restriction enzyme ScrFI, to give the vector pCSY226-NLS-IκBα.

### Example 2

### Construction of Plasmids Able to Express the Fusion Proteins GFP-β-TrCP and GFP-NLS-β-TrCP in Yeast

The following plasmids express derivatives of the human β-TrCP protein fused with a variant of Green Fluorescent Protein (GFP) from Aequora victoria, in the yeast Saccharomyces cerevisiae. Depending on the plasmid construction, the fusion proteins do or do not contain the nuclear localisation sequence from the big-T antigen of the SV40 virus. The introduction of this sequence will cause proteins that contain it to be directed to the nuclear compartment of the cell.

A 620 base-pair (bp) fragment corresponding to the GAL1 gene promoter (pGAL1) of the yeast Saccharomyces cerevisiae was amplified by Polymerase Chain Reaction (PCR) from the genomic DNA of a wild type S. cerevisiae strain, X2180-1A, using oligonucleotides “pGAL1(Asp)Forw”, sequence

5′-GCTGGGTACCTTAATAATCATATTACATGGCATTA-3′ [SEQ ID N^{o}6] and “pGAL1(EcoRI)Rev”, sequence

5′-GGCGGAATTCTATAGTTTTTTCTCCTTGACGTTA-3′ [SEQ ID N^{o}7].

The resulting fragment was digested with restriction enzymes Asp7181 and EcoRI and inserted into the S. cerevisiae-E. coli shuttle plasmid pRS306, previously digested with the enzymes Asp718I and EcoRI, to produce the vector pRS306-pGAL1.

A 720 base-pair (bp) fragment from vector pUC19-yEGFP3, and corresponding to a variant of the gene coding for the Green Fluorescent Protein (GFP) of Aequora victoria, in which the sequence had been optimised for expression in yeast (yEGFP3), was amplified by Polymerase Chain Reaction (PCR), using the oligonucleotides “GFPEcoR15′”, sequence

5′-GGTCGGAATTCATGTCTAAAGGTGAAGAATTATTC-3′ [SEQ ID N^{o}8] and “GFPBamHI(SmaI/SrfI PstI)3′”, sequence

5′-GGCGGGATCCGCCCGGGCTCTGCAGTTTGTACAATTCATCCATACC-3′ [SEQ ID N9].

The resulting fragment was digested with restriction enzymes BamHI and EcoRi and inserted into plasmid pRS306-pGALI, previously digested with the enzymes BamHI and EcoRl, to produce the vector pRS306-pGAL1-yEGFP3.

A 340 base-pair (bp) fragment corresponding to the ADHI gene promoter (tADH1) of the yeast Saccharomyces cerevisiae was amplified by Polymerase Chain Reaction (PCR) from the genomic DNA of a wild type S. cerevisiae strain, X2180-1A, using oligonucleotides “TermADH1(NotIBstXI)5′”, sequence 5′-GGCGGCGGCCGCCACCGCGGTGGGCGAATTTCTTATGATTTATG-3′ [SEQ ID N^{o}10] and “TermADH1(SacI)3′”, sequence 5′-GGCGGAGCTCTGGAAGAACGATTACAACAG-3′ [SEQ ID N^{o}11].

The resulting fragment was digested with restriction enzymes SacI and NotI and inserted into plasmid pRS306-pGAL1-yEGFP3, previously digested with the enzymes SacI and NotI, to produce the vector pCSY226. The gene coding for the βTrCP protein was purified from the plasmid pGad 1318-βTrCP by digestion with the restriction enzymes BamHI and NotI. The fragment was cloned in the plasmid pCSY226 prepared by digestion with the restriction enzymes BamHI and NotI. The resulting vector has been called pCSY226-βTrCP.

A version of this vector also includes the nuclear location sequence NLS. This was obtained by synthesising a pair of oligonucleotides complementary to the sequences “NLS-5′”: 5′-ACCTCCAAAAAAGAAGAGAAAGGTCGAATT-3′ [SEQ ID N^{o}12], and “NLS-3′”: 5′-AATTCGACCTTTCTCTTCTTlTTTGGAGGT-3′ [SEQ ID N^{o}26]and rehybridising them to form a double-stranded DNA. This DNA fragment was then incorporated into the vector pCSY226-βTrCP digested with restriction enzyme ScrFI, to give the vector pCSY226-NLS-βTrCP.

### Example 3

### Construction of Plasmids Able to Express the Fusion Proteins GFP-β-TrCP and GFP-NLS-β-TrCP in Yeast

The following plasmids express, in the yeast Saccharomyces cerevisiae, derivatives of the human β-TrCP protein containing a repetition of three antigenic Flag motifs at their amino-terminal end. The expression of these fusion proteins is induced by growing the yeast cells containing plasmid for 1 to 10 hours in culture medium containing 2 to 5% galactose.

A 700 base-pair (bp) fragment corresponding to the PGK1 gene promoter (pPGK1) of the yeast Saccharomyces cerevisiae was amplified by Polymerase Chain Reaction (PCR) from the genomic DNA of a wild type S. cerevisiae strain, X2180-1A, using oligonucleotides “pPGK1-Asp718-5′”, sequence 5′-GGCGGGTACCGTGAGTAAGGAAAGAGTGAGG-3′ [SEQ ID N^{o}13] and “pPGK-EcoRI-3′”, sequence 5′-GGCGGAATTCTGTTTTATATTTGTTGTAAAAAG-3′ [SEQ ID N^{o}14].

The resulting fragment was digested with restriction enzymes Asp718I and EcoRI and inserted into the S. cerevisiae-E. coli shuttle plasmid pRS304, previously digested with the enzymes Asp718I and EcoRI, to produce the vector pRS304-pPGK1.

A 100 base-pair (bp) fragment corresponding a string of 3 FLAG reporter sequences (3FLAG) was amplified by Polymerase Chain Reaction (PCR) from the vector p3XFLAG-myc-CMV-24 5Sigma Aldrich, using oligonucleotides “3FLAG-EcoRI-5′”, sequence 5′-GGCGGAATTCATGGACTACAAAGACCATGACGG-3′ [SEQ ID N^{o}15] and “3FLAGBamHI(SmaI/SrfI PstI)3′”, sequence 5′-GGCGGGATCCGCCCGGGCTCTGCAGCTTGTCATCGTCATCCTTGTA-3′ [SEQ ID N^{o}16].

The resulting fragment was digested with restriction enzymes BamHI and EcoRI and inserted into plasmid pRS304-pPGK1, previously digested with the enzymes BamHI and EcoRI, to produce the vector pRS304-pPGK1-3FLAG.

A 340 base-pair (bp) fragment corresponding to the ADH1 gene terminator signal (tADH1) of the yeast Saccharornyces cerevisiae was amplified by Polymerase Chain Reaction (PCR) from the genomic DNA of a wild type S. cerevisiae strain, X2180-1A, using oligonucleotides “TermADH1 (NotIBstXI)5′”, sequence5′-GGCGGCGGCCGCCACCGCGGTGGGCGAATTTCTTATGATTTATG-3′[SEQ ID N^{o}10] and “TermADH1(SacI)3′”, sequence 5′-GGCGGAGCTCTGGAAGAACGATTACAACAG-3′ [SEQ ID N^{o}11].

The resulting fragment was digested with restriction enzymes SacI and NotI and inserted into plasmid pRS304-pPGK1-3FLAG, previously digested with the enzymes SacI and NotI, to produce the vector pCSY614.

The gene coding for the TrCP protein was purified from the plasmid pGad1318-βTrCP by digestion with the restriction enzymes BamHI and NotI. The fragment was cloned in the plasmid pCSY614 prepared by digestion with the restriction enzymes BamHI and NotI. The resulting vector has been called pCSY614-βTrCP.

A version of this vector also includes the nuclear location sequence NLS. This was obtained by synthesising a complementary pair of oligonucleotides, for the sequence 5′ACCTCCAAAAAAGAAGAGAAAGGTCGAATT-3′ [SEQ ID N^{o}12] and rehybridising them to form a double-stranded DNA. This DNA fragment was then incorporated into the vector pCSY614-βTrCP digested with restriction enzyme ScrFI, to give the vector pCSY614-NLS-βTrCP.

### Examples 4 to 12

### Development of the Screening Method According to the Invention

### Example 4

### Interaction Between Yeast Skp1 and Human β-TrCP Proteins in Yeast Cells

The interaction between Skp1 and β-TrCP proteins is visualised using the two-hybrid method Bartel et al. (in Cellular Interactions in Development: a practical approach (1991), Oxford University Press, Oxford, pp153-179). Yeast cells are simultaneously transformed with the pGAD-βTrCP plasmid which expresses the human β-TrCP protein fused with the activator domain Gal4, with the plasmid pLexSkp1-1 which expresses the yeast protein Skp1 fused to the DNA-binding domain of the bacterial protein LexA, and with the plasmid pSH18-34 which includes the LacZ reporter gene coding for β-galactosidase, under the control of LexA operators. Measurement of β-galactosidase activity in cellular extracts from such cells shows that expression of this reporter gene increases by a factor of 15 when compared to its expression in cells expressing only one of the two fusion proteins described herein. This induction of reporter gene expression indicates that the Skp1 protein from Saccharomyces cerevisiae is capable of interacting with the human β-TrCP protein. β-galactosidase activity is expressed in nmoles of substrate transformed per minute per mg of protein (nmole/min/mg).

### Example 5

### Localisation in Yeast Cells of Human Proteins IκBα and β-TrCP According to Whether or not They are Fused to an NLS Sequence of SV40

The yeast cells containing the plasmids able to express the hybrid proteins, either GPF-IκBα, GFP-NLS-IκBα, GFP-β-TrCP, or GFP-NLS-β-TrCP under the GAL1 promoter, are grown in the presence of 2% galactose for 2 hours and then observed with a fluorescence microscope. The position of the nucleus is revealed using a nuclear-specific dye, Hoescht 333-42.

### Example 6

### Phosphorylation of the IκBα Protein in Yeast Cell Nuclei

Example 6 shows how presence of the human IκBα protein in the nuclei of yeast cells leads to its phosphorylation at serines 32 and 36.

Cells expressing either the fusion protein GFP-IκBα or tripartite fusion protein GFP-NLS-IκBα under the GAL1 promoter, are grown in Minimum Essential Medium in the presence of 2% galactose for 2 hours. The proteins from these cells are then extracted according to the protocol described by Kuras et al. (Mol. Cell (2002), 10:69-80). The proteins are then analysed by Western blotting firstly using a specific antibody to the GFP protein (called “GFP-IκBα”) and secondly an antibody which specifically recognises human IκBα protein phosphorylated at serine 32 (called “P-IκBα”). As a control for the total amount of protein loaded in each well, the same proteins are analysed with an antibody specific for yeast Lysy1-tRNA-synthase (called “LysRS”). The proteins made by the parental strain of yeast which does not express any fusion protein (called “control”) serve as a test for specificity.

### Example 7

### Degradation of the GFP-NLS-IκBα Protein

Example 7 shows, by epifluorescent microscopy, the degradation of the tripartite fusion protein GFP-NLS-IκBα in the yeast cells which, at the same time, express the tripartite fusion protein Flag-NLS-β-TrCP.

All the strains used are grown and analysed by fluorescence microscopy in an identical manner. The cells are grown for 120 minutes in galactose-rich medium as the source of carbon. At time t=0, 2% glucose is added to the culture and the cells are observed by epifluorescent microscope (Nikon Eclipse fluorescent microscope equipped with an Omega XF116 filter). All the images were recorded using a Hamamastu® camera identically adjusted and analysed with LUCIA G software, just before (t=0) and 10, 20, 30 and 60 minutes after addition of the glucose. The fluorescence of the fusion proteins GFP-IκBα or GFP-NLS-IκBα is called “GFP”. The position of the nucleus (called “DNA”) in the cells was revealed using a nuclear-specific dye, Hoescht 333-42.

A) yeast strain CYS22 (MATa, his3, leu2, trp1, ura3::pGAL1-GFP-IκBα::URA3) expressing the fusion protein GFP-IκBα without NLS and localised in the cytoplasm of yeast cells;

B) yeast strain CYS61 (MATa, his3, leu2, ura3::pGAL1-GFP-IκBα::URA3, trp1:.pGAL1-3Flag-βTrCP::TRP1) expressing the fusion proteins GFP-IκBα and Flag-β-TrCP, localised in the cytoplasm of yeast cells;

C) yeast strain CYS126 (MATa, his3, leu2, trp1, ura3::pGAL1-GFP-NLS-IκBα::URA3) expressing the fusion protein GFP-NLS-IκBα localised in the nucleus of yeast cells;

D) yeast strain CYS135 (MATa, his3, leu2, ura3::pGAL1-GFP-NLS-IκBα::URA3, trp1::pGAL1-3Flag-NLS-βTrCP::TRP1) expressing the fusion proteins GFP-NLS-IκBα and Flag-NLS-β-TrCP, localised in the nucleus of yeast cells.

### Example 8

### Degradation of GFP-NLS-IκBα With or Without Co-Expression of Flag-NLS-β-TrCP (Results from Fluorescence)

Example 8 shows, by measurement of the fluorescence produced, the degradation of the tripartite fusion protein GFP-NLS-IκBα in the yeast cells which, at the same time, do or do not express the tripartite fusion protein Flag-NLS-β-TrCP.

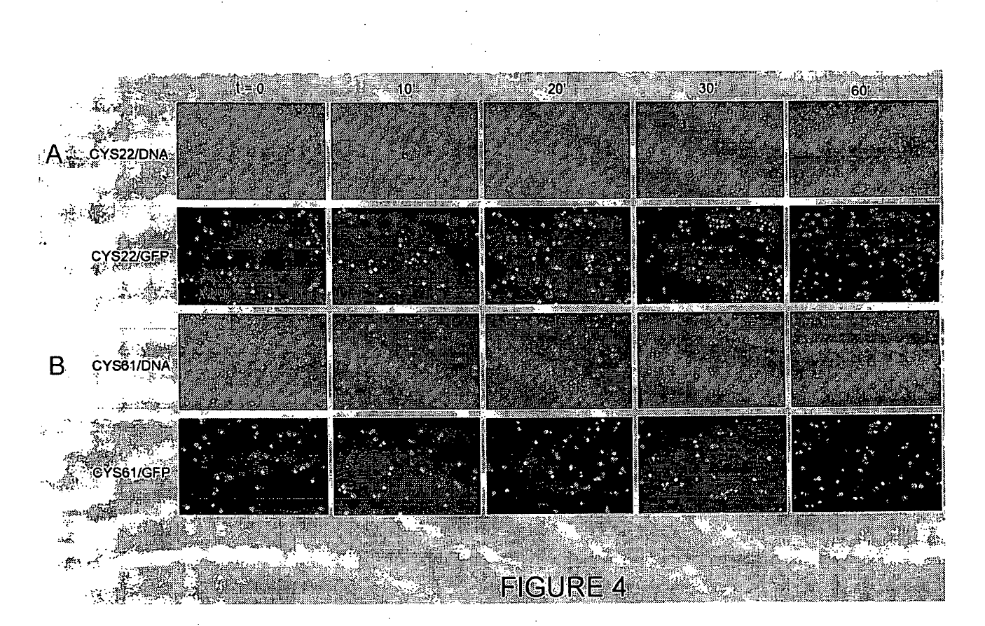

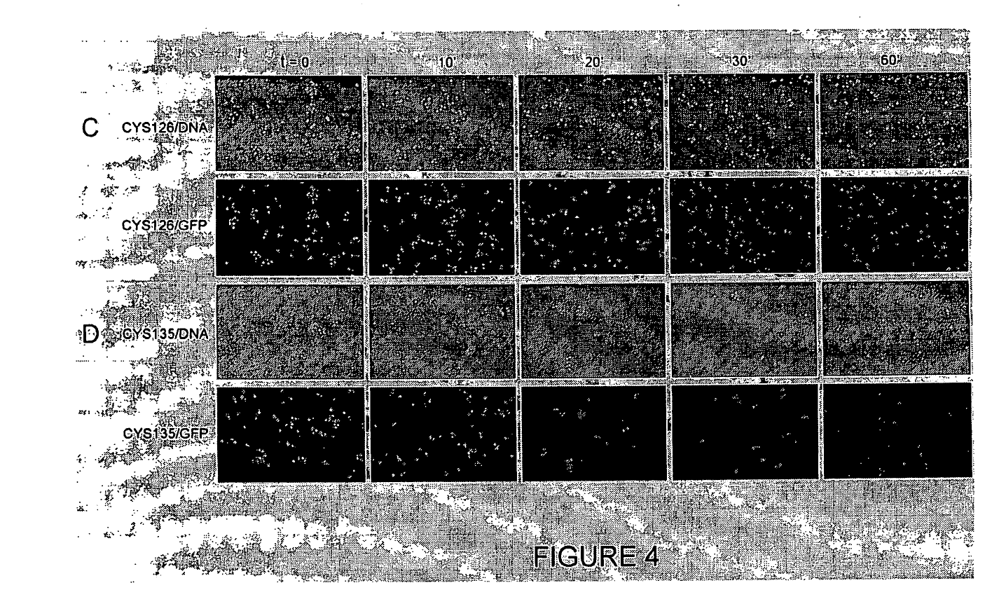

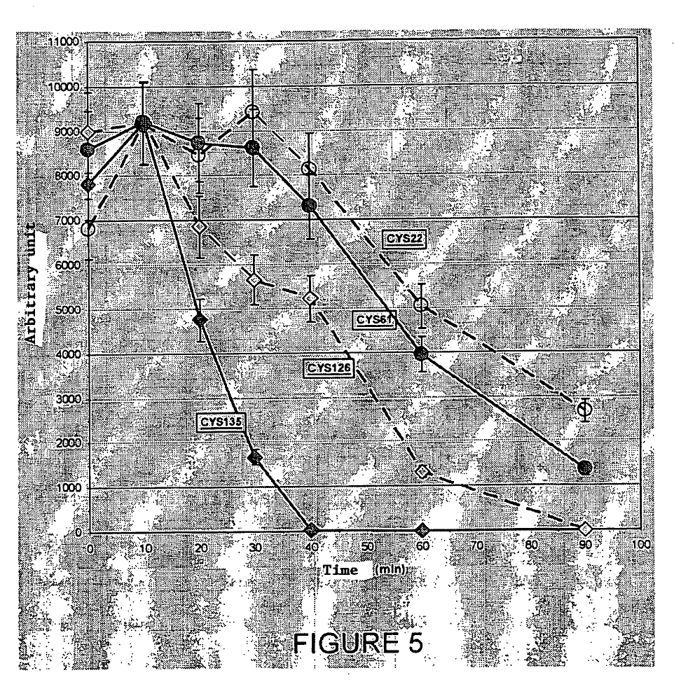

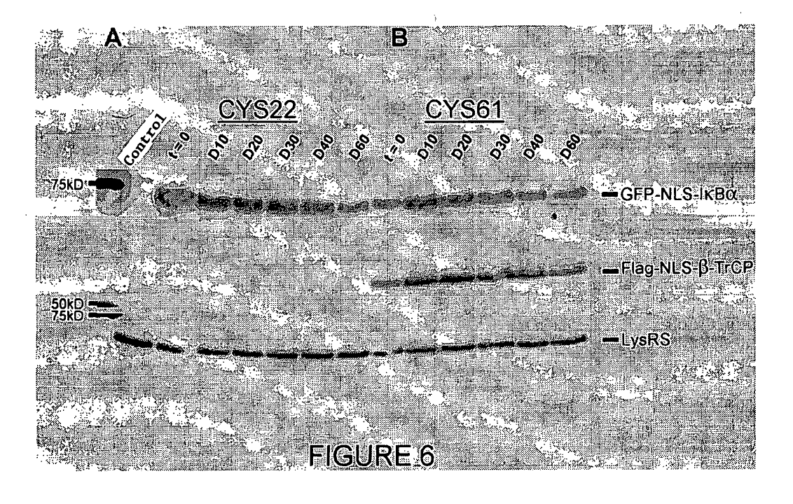

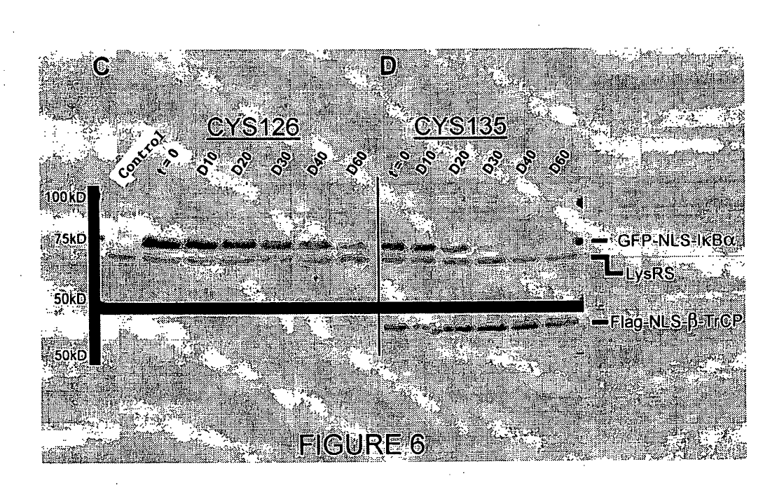

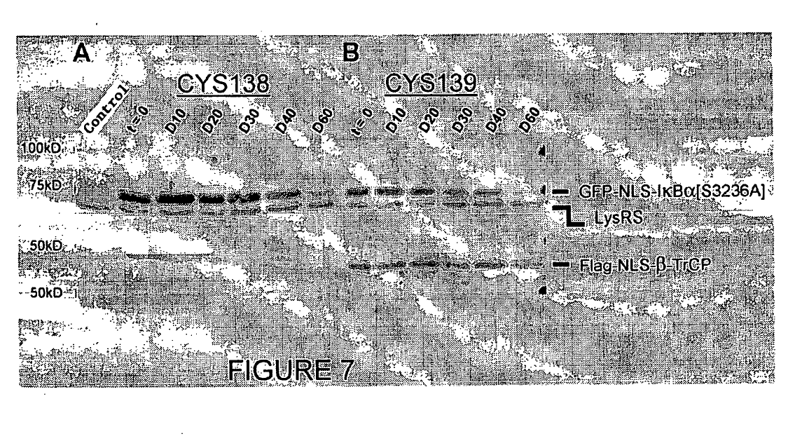

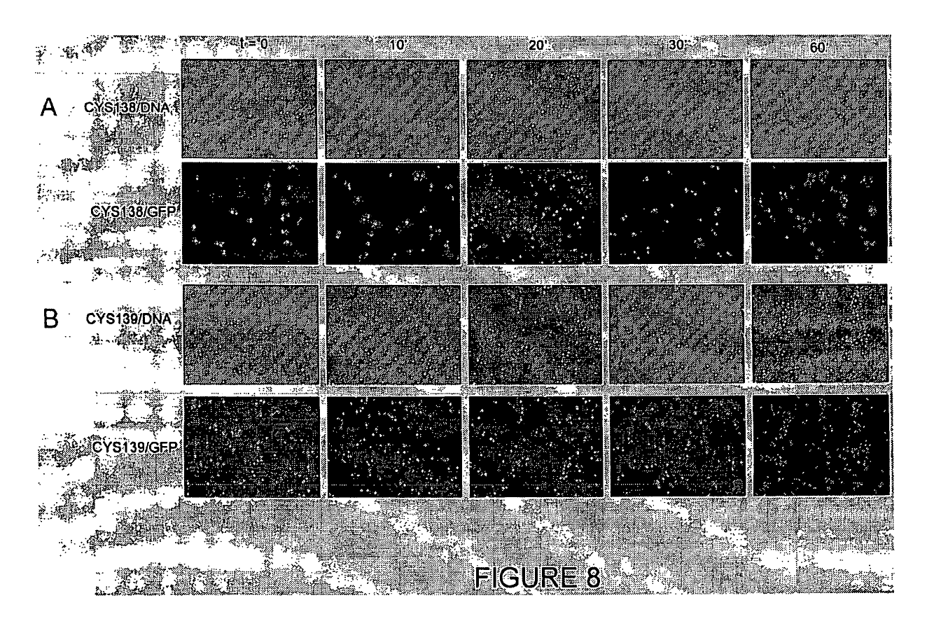

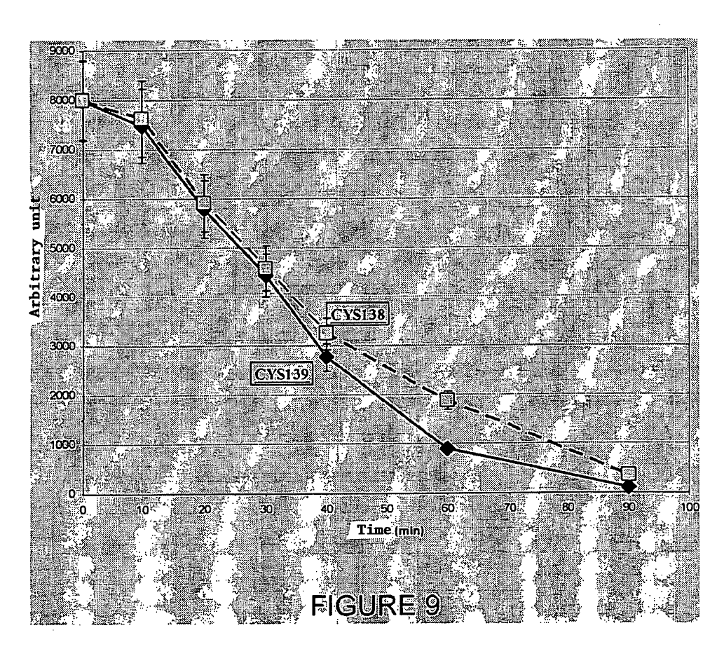

Strains of yeast identical to those described in FIG. 4, and grown under the same conditions as described in FIG. 4, were analysed by fluorescence microscopy. For each strain, the fluorescence of 200 cells (at least) was measured just before (t=0) and 10, 20, 30 and 60 minutes after the addition of glucose, using the LUCIA G software. The results are given, in arbitrary units, as the amount of fluorescence measured per cell.

### Example 9

### Degradation of GFP-NLS-IκBα with or Without Co-Expression of Flag-NLS-β-TrCP (Results from Immunoblotting)

Example 9 shows, by Western Blot type biochemical analysis, the degradation of the tripartite fusion protein GFP-NLS-IκBα in yeast cells which, at the same time, express the tripartite fusion protein Flag-NLS-β-TrCP. All the strains used were grown and analysed in an identical manner. The cells were grown for 120 minutes in galactose-rich medium as the source of carbon. At time t=0, 2% glucose is added to the culture and the total protein is extracted just before (t=0) and 10, 20, 30 and 60 minutes after the addition of glucose. These proteins are analysed by Western blotting using an antibody to the GFP part of the fusion proteins including IκBα (called “GFP-NLS-IκBα”) and an antibody to the Flag part of the fusion protein Flag-NLS-β-TrCP (called “Flag-NLS-β-TrCP”). As a control for the total amount of protein loaded in each well, the same proteins are analysed with an antibody specific for yeast Lysyl-tRNA-synthase (called “LysRS”). The proteins made by the parental strain of yeast which does not express any fusion protein (called “control”) serve as a test for specificity.

A) yeast strain CYS22 (MATα, his3, leu2, trp1, ura3::pGAL1-GFP-IκBα::URA3) expressing the fusion protein GFP-IκBα without NLS and localised in the cytoplasm of yeast cells;

B) yeast strain CYS61 (MATa, his3, leu2, ura3::pGAL1-GFP-IκBα::URA3, trp1::pGAL1-3Flag-βTrCP::TRP1) expressing the fusion proteins GFP-IκBα and Flag-β-TrCP, localised in the cytoplasm of yeast cells;

C) yeast strain CYS126 (MATa, his3, leu2, trp1, ura3::pGAL1-GFP-NLS-IκBα::URA3) expressing the fusion protein GFP-NLS-IκBα localised in the nucleus of yeast cells;

D) yeast strain CYS135 (MATa, his3, leu2, ura3::pGAL1-GFP-NLS-IκBα::URA3, trp1::pGAL1-3Flag-NLS-βTrCP::TRP1) expressing the fusion proteins GFP-NLS-IκBα and Flag-NLS-β-TrCP, localised in the nucleus of yeast cells.

### Example 10

### Degradation of GFP-NLS-IκBα Mutated at Serine Residues 32 and 36, With or Without Co-Expression of Flag-NLS-D-TrCP (Results From Immunoblotting)

Example 10 shows, by Western Blot type biochemical analysis, the degradation of the mutated tripartite fusion protein GFP-NLS-Iκα[S3236A] in which the phosphorylation sites Ser32 and Ser36 have been replaced by Ala residues, mutations that, in human cells, make the protein non-degradable. Analysis was carried out also in yeast cells either expressing or not expressing the tripartite fusion protein Flag-NLS-β-TrCP. All the strains used were grown and analysed in an identical manner. The cells were grown for 120 minutes in galactose-rich medium as the source of carbon. At time t=0, 2% glucose is added to the culture and the total protein extracted just before (t=0) and 10, 20, 30 and 60 minutes after the addition of glucose. These proteins are analysed by Western blotting using an antibody to the GFP part of the fusion proteins including IκBα[S3236A] (called “GFP-NLS-IκBα[S3236A]”) and an antibody to the Flag part of the fusion protein Flag-NLS-β-TrCP (called “Flag-NLS-β-TrCP”). As a control for the total amount of protein loaded in each well, the same proteins are analysed with an antibody specific for yeast Lysyl-tRNA-synthase (called “LysRS”). The proteins made by the parental strain of yeast which does not express any fusion protein (called “control”) serve as a test for specificity.

A) yeast strain CYS138 (MATα, his3, leu2, trp1, ura3::pGAL1-GFP-NLS-IκBα[S3236A]::URA3) expressing the mutated fusion protein GFP-NLS-IκBα[S3236A] localised in the nucleus of yeast cells;

B) yeast strain CYS139 (MATα, his3, leu2, ura3:.pGAL1-GFP-NLS-IκBα[S3236A]::URA3, trp1::pGAL1-3Flag-NLS-βTrCP::TRP1) expressing the fusion proteins GFP-NLS-IκBα[S3236A] and Flag-NLS-β-TrCP, localised in the nucleus of yeast cells.

### Example 1

### Degradation of GFP-NLS-IκBα with or Without Co-Expression of Flag-NLS-β-TrCP (Results from Fluorescence)

Example 11 shows, by epifluorescent microscopy, the degradation of the tripartite fusion protein GFP-NLS-IκBα[S3236A] in the yeast cells which, at the same time, express the tripartite fusion protein Flag-NLS-β-TrCP. The 2 strains used (CYS138 and CYS139) were grown, and analysed by fluorescence microscopy, in an identical manner. The cells are observed by epifluorescent microscopy (Nikon Eclipse fluorescent microscope equipped with an Omega XF116 filter). All the images were recorded using a Hamamastu® camera identically adjusted and analysed with LUCIA G software, just before (t=0) and 10, 20, 30 and 60 minutes after addition of the glucose. The fluorescence of the fusion proteins GFP-IκBα or GFP-NLS-IκBα is called “GFP”. The position of the nucleus (called “DNA”) in the cells is revealed using a nuclear-specific dye, Hoescht 333-42.

### Example 12

### Degradation of GFP-NLS-IκBα with or Without Co-Expression of Flag-NLS-β-TrCP (Results from Fluorescence)

Example 12 shows, by measurement of the fluorescence emitted, the degradation of the tripartite fusion protein GFP-NLS-IκKα[S3236A] in the strains of yeast described herein. For each strain, the fluorescence of 200 cells (at least) was measured just before (t=0) and 10, 20, 30 and 60 minutes after the addition of glucose, using the LUCIA G software. The results are given, in arbitrary units, as the amount of fluorescence measured per cell.

TABLE 1
Genotype of the strains of yeast Saccharomyces cerevisiae prepared for
use in the present invention.

| Strain | Genotype |
|---|---|
| CC788-2B | MATa, his3, leu2, ura3, trp1. |
| CYS22 | MATa, his3, leu2, trp1, ura3::pGAL1-GFP-IκBα::URA3 |
| CYS61 | MATa, his3, leu2, ura3::pGAL1-GFP-IκBα::URA3, |
| | trp1::pGAL1-3Flag-βTrCP::TRP1 |
| CYS122 | MATa, his3, leu2, trp1, ura3::pGAL1-GFP-βTrCP::URA3 |
| CYS123 | MATa, his3, leu2, trp1, ura3::pGAL1-GFP- |
| | NLS-βTrCP::URA3 |
| CYS126 | MATa, his3, leu2, trp1, ura3::pGAL1-GFP-NLS-IκBα::URA3 |
| CYS135 | MATa, his3, leu2, ura3::pGAL1-GFP-NLS-IκBα::URA3, |
| | trp1::pGAL1-3Flag-NLS-βTrCP::TRP1 |
| CYS138 | MATa, his3, leu2, trp1, ura3::pGAL1-GFP-NLS- |
| | IκBα[S3236A]::URA3 |
| CYS139 | MATa, his3, leu2, ura3::pGAL1-GFP-NLS-IκBα[S3236A] |
| | ::URA3, trp1::pGAL1-3Flag-NLS-βTrCP::TRP1 |

TABLE 2
(SEQUENCES)

| SEQ ID N^{o} | Type | Description |
|---|---|---|
| 1 | DNA | GFP-NLS-IkBα |
| 2 | Protein | GFP-NLS-IkBα |
| 3 | DNA | GFP-NLS-βTrCP |
| 4 | Protein | GFP-NLS-βTrCP |
| 5 | DNA | NLS sequence of the SV40 |
| | | big-T antigen |
| 6-16 | DNA | Primers |
| 17 | Protein | HA antigen |
| 18 | Protein | FLAG monomer |
| 19 | Protein | FLAG trimer |
| 20 | Protein | Nucleoplasmin NLS |
| 21 | Protein | NLS repressor alpha 2 (1) |
| 22 | Protein | NLS repressor alpha 2 (2) |
| 23 | Protein | Gal4 NLS |
| 24 | DNA | SV40 T-Ag NLS |
| 25 | DNA | Primer |

TABLE 3
List of GFPs usable according to the invention

| | | | | | | | | | | | | | | | | | | | | | | λ | λ | |
|---|---|---|---|---|---|---|---|---|---|---|---|---|---|---|---|---|---|---|---|---|---|---|---|---|
| | Residues | | | | | | | | | | | | | | | | | | | | | excitation | emission | |
| | 26 | 46 | 64 | 65 | 66 | 67 | 68 | 69 | 70 | 72 | 80 | 145 | 146 | 153 | 163 | 164 | 167 | 168 | 175 | 203 | 212 | (nm) | (nm) | Références |
| wtGFP | Lys | Phe | Phe | Ser | Tyr | Gly | Val | Gln | Cys | Ser | Gln | Tyr | Asn | Met | Val | Asn | Ile | Ile | Ser | Thr | Asn | 395-470 | 509-540 | Heim et al., 1994 |
| BFP | | | Leu | | His | | | | | | | | | | Ala | | | | | | | 387 | 450 | Quantum |
| CFP | | | Leu | | Trp | | | | | | | | Ile | Thr | Ala | His | | | | | | 436 | 480 | |
| (YRC) | | | | | | | | | | | | | | | | | | | | | | | | |
| EBFP | | | Lys | Thr | His | | | | | | | Phe | | | | | | | | | | 380 | 440 | Yang et al., 1996 |
| (Clontech) | | | | | | | | | | | | | | | | | | | | | | | | Cormack et al., 1996 |
| ECFP | | | Leu | Thr | | | | | | | | | Ile | Thr | Ala | | | | | | | 475 | 501 | Heim et al., 1994-1996 |
| (Clontech) | | | | | | | | | | | | | | | | | | | | | | | | |
| ECFP | Arg | | Lys | Thr | Trp | | | | | | | | Ile | Thr | Ala | His | | | | | Lys | 434 | 474 | Miyawaki et al., |
| | | | | | | | | | | | | | | | | | | | | | | | | 1997 |
| EGFP = | | | Leu | Thr | | | | | | | | | | | | | | | | | | 488 | 508 | Yang et al., 1998 |
| GFPmut1 | | | | | | | | | | | | | | | | | | | | | | | | |
| (Clontech) | | | | | | | | | | | | | | | | | | | | | | | | Cormack et al., 1996 |
| EYFP | | | | Gly | | | Leu | | | Ala | | | | | | | | | | Tyr | | 514 | 527 | Ormö et al., 1997 |
| (Clontech) | | | | | | | | | | | | | | | | | | | | | | | | |
| GFP405 | | | | | | | | | | | | | | | | | | | | | | 405 | 510 | Clontech |
| | | | | | | | | | | | | | | | | | | | | | | | | “SuperBright” |
| GFPmut3 | | | | Gly | | | | | | Ala | | | | | | | | | | | | 501 | 511 | Cormack et al., 1996 |
| GFPuv | | | | | | | | | | | | | | | | | | | | | | 395 | 408 | Crameri et al., 1996 |
| mCFP | | | | | Trp | | | | | | | | | | Ala | | | | Gly | | | 440 | 485 | Haseloff et al., 1999 |
| mGFP5 | | | | | | | | | | | | | | | Ala | | Thr | | Gly | | | 400-475 | 508 | Haseloff et al., 1997 |
| | | | | | | | | | | | | | | | | | | | | | | | | Siemering et al., 1996 |
| mYFP | | | | Gly | | | | | | Ala | | | | | Ala | | Tyr | | Gly | Tyr | | 514 | 527 | Haseloff et al., 1999 |
| PA-GFP | | | | | | | | | | | | | | | Ala | | | | | His | | 413-488 | 520 | Patterson et al., 2002 |
| rsGFP | | | Leu | Cys | | | | | | | | | | | | | | Thr | | | | 473 | 509 | Quantum |
| RsGFP | | | | Gly | | | | | | Ala | | | | | | | | Trp | | | | 505 | 522 | Reed et al., 2001 |
| S65T | | | | Thr | | | | | | | | | | | | | | | | | | 488 | 507 | Heim et al., 1995 |
| T-Sapphire | | | | | | | | Met | Val | | | | | | Ala | | | | Gly | Ile | | 399 | 511 | O. Zapata-Hommer |
| | | | | | | | | | | | | | | | | | | | | | | | | and O. Griesbeck, |
| | | | | | | | | | | | | | | | | | | | | | | | | 2003 |
| yEGFP3 | | | | Gly | | | | | | Ala | | | | | | | | | | | | 501 | 511 | Cormack et al., 1997 |
| (Cormack) | | | | | | | | | | | | | | | | | | | | | | | | |
| YFP | | | | Gly | | | | | | Ala | | | | | | | | | | Tyr | | 500 | 535 | Ormö et al., 1996 |
| (YRC) | | | | | | | | | | | | | | | | | | | | | | | | |
| YFP- | | | | Gly | | | Leu | Met | | Ala | | | | | | | | | | Tyr | | 490-510 | 515 | Griesbeck et al., 2001 |
| citrine | | | | | | | | | | | | | | | | | | | | | | | | |
| YFP-Venus | | Leu | Leu | Gly | | | Leu | | | Ala | | | | Thr | Ala | | | | Gly | Tyr | | 488 | 514-527 | Nagai et al., 2002 |
| (YRC) | | | | | | | | | | | | | | | | | | | | | | | | |

### REFERENCES

Arroyo et al. (Unpublished (1996), direct submission to MIPS).

Ausubel et al. (in Current Protocols in Molecular Biology, (1990-2004), John Wiley and Sons Inc, N.Y.).

Bailis et al., Genetics (1990), 126:535-547)

Ballard, Immunol Res. (2001), 23:157-166

Bartel et al. (in Cellular Interactions in Development: a practical approach (1991), Oxford University Press, Oxford, pp 153-179).

Baud and Karin, Trends Cell Biol. (2001), 11:372-377

Ben Neriah, Nat Immunol. (2002), 3:20-26

Bennetzen and Hall (J. Biol. Chem. (1982) 257(6): 3018-3025).

Bolle et al. (Yeast (1992) 8(3): 205-213).

Cornack et al. (Gene (1996) 173 (1): 33-38).

Hanes and Brent (Cell (1989), 57:1275-1293).

Hanes and Brent (Cell (1989), 57:1275-1293)

Hay et al., Philos. Trans. R. Soc. Lond. B. Biol. Sci. (1999) 354:1601-1609

Johnston and Davis (Mol. Cell. Biol. (1984) 4 (8): 1440-1448).

Karin et al. (PNAS (1984) 81(2): 337-341).

Kerjan et al. (Nucleic Acids Res.(1986) 14(20): 7861-7871).

Kroll et al., J. Biol. Chem. (1999), 274:7941-7945

Kroll et al., J. Biol. Chem. (1999), 274:7941-7945

Kuras et al. (EMBO J. (1996) 15(10): 2519-2529).

Kuras et al. (Mol. Cell (2002), 10:69-80).

Lewis and Manning, Curr. Opin. Chem. Biol. (1999) 3: 489-494

Magnani et al., Curr. Drug Targets. (2000) 1:387-99

Margottin et al. (Molec. Cell (1998), 1:565-574).

Margottin et al. (Molec. Cell (1998), 1:565-574).

Nourani et al., Mol. Cell. Biol. (1997), 20:7881-7892).

Patton et al. (Genes & Dev (1998), 12:692-705)

Patton et al. Genes & Dev (1998), 12:692-705.

Rad et al. (Yeast (1991) 7(5): 533-538).

Rothstein, in Methods in Enzymnology, (1991), 194:281-301

Sambrook et al. (in Molecular Cloning, Laboratory Manual, 2^{nd} edition, (1989), Cold Spring Harbor, N.Y.)

Schiestl et al. (Curr. Genet. (1989), 16:339-346

Sherman et al., in Methods in Yeast Genetics: a Laboratory Manual, (1979), Cold Spring Harbor, N.Y.

Sikorski and Hieter (Genetics (1989) 122(1): 19-27).

Strausberg et al. (PNAS (1999), 99(26): 16899-16903

Thomas et al. (J. Biol. Chem. (2000) 275(52): 40718-40724).

Vidal et al., Trends Biotechnol., (1999), 17:374-381

Winston et al., Genes Dev. (1999), 13:270-283

Winston et al., Genes Dev. (1999), 13:270-283

Yaron et al. (Nature (1998) 396(6711): 590-594).

1. A method for screening agents modulating IκBα protein ubiquitination by a functional ubiquitin ligase protein complex containing β-TrCP protein, said method comprising the following steps:
(a) bringing into contact a candidate agent to be tested with recombinant yeast cells that express in their nucleus:
(i) a fusion protein containing the polypeptide IκBα and at least one first detectable protein; and
(ii) a protein containing the polypeptide β-TrCP;
(b) quantifying said first detectable protein in the yeast cells, at the end of at least one predetermined period of time after bringing the candidate agent into contact with said cells;
(c) comparing the result obtained in step (b) with a control result obtained when step (a) is carried out in the absence of the candidate agent.

2. A method according to claim 1, characterised in that step (a) includes the following steps:
(a1) growing yeast cells which express in their nucleus a fusion protein containing the polypeptide IκBα and at least one first detectable protein;
(a2) stopping the expression of said fusion protein containing the polypeptide IκBα and at least one first protein detectable by the yeast cells;
(a3) bringing the yeast cells obtained at the end of step (a2) into contact with the candidate agent to be tested.

3. A method according to claim 2, characterised in that the yeast cells express the protein containing the polypeptide β-TrCP throughout all the steps (a1), (a2) and (a3).

4. A method according to claim 2, characterised in that the yeast cells express the protein containing the polypeptide β-TrCP throughout all the steps (a2) and (a3) and do not express the protein containing the polypeptide β-TrCP in step (a1).

5. A method according to claim 2, characterised in that the yeast cells express the protein containing the polypeptide β-TrCP throughout all the steps (a2) and (a3), and
(i) do not express the protein containing the polypeptide β-TrCP for a predetermined time at the start of step (a1);
(ii) do express the protein containing the polypeptide β-TrCP for the remainder of step (a1).

6. A method according to claim 1, characterised in that the detectable protein in the polypeptide containing IκBα polypeptide is chosen from an antigen, a fluorescent protein and a protein having enzymatic activity.

7. A method according to claim 6 characterised in that the detectable protein is a fluorescent protein selected from the GFP protein or one of its derivatives, the YFP protein or one of its derivatives, and the dsRED protein.

8. A method according to claim 6, characterised in that the detectable protein is a protein having enzymatic activity selected from luciferase and β-lactamase.

9. A method according to claim 6, characterised in that the detectable protein is an antigen selected from the Ha peptide and the Flag peptide.

10. A method according to claim 1, characterised in that the protein containing the polypeptide β-TrCP a fusion protein also containing a second detectable protein.

11. A method according to claim 10, characterised in that the second detectable protein included in the fusion protein containing the β-TrCP polypeptide is chosen from an antigen, a fluorescent protein and a protein having enzymatic activity.

12. A method according to claim 10, characterised in that (i) the first detectable protein included in the fusion protein containing the polypeptide IκBα and (ii) the second detectable protein included in the fusion protein containing the polypeptide β-TrCP are different from each other.

13. A method according to claim 1, characterised in that the protein containing the polypeptide IκBα also contains a nuclear localisation peptide.

14. A method according to claim 1, characterised in that the protein containing the polypeptide β-TrCP also contains a nuclear localisation peptide.

15. A method according to claim 1, characterised in that the protein containing the polypeptide IκBα is the protein with the sequence SEQ ID N^{o}2.

16. A method according to claim 1, characterised in that the protein containing the polypeptide β-TrCP is the protein with the sequence SEQ ID 4.

17. A method according to claim 1, characterised in that at step (b), when the first detectable protein is an antigen, said first detectable protein is quantified by detecting the complexes formed between said protein and the antibodies which recognise it.

18. A method according to claim 1, characterised in that at step (b), when the first detectable protein is a fluorescent protein, said detectable protein is quantified by measuring the fluorescent signal emitted by said protein.

19. A method according to claim 1, characterised in that at step (b), when the first detectable protein is a protein having enzymatic activity, said detectable protein is quantified by measuring the quantity of substrate modified by said protein.

20. A method according to claim 1, characterised in that the recombinant yeast cells are transformed with: respectively:
(1) a first polynucleotide that contains (a) an open reading frame coding for (i) the fusion protein containing, the IκBα polypeptide and (iii) a first detectable protein, and a regulatory sequence functional in yeast cells which controls expression of said open reading frame; and
(2) a second polynucleotide that contains (a) an open reading frame coding for (i) the protein containing the β-TrCP polypeptide, ii) a nuclear localisation sequence and (iii) a regulatory sequence functional in yeast cells which controls expression of said open reading frame;

21. A method according to claim 20, characterised in that the regulatory sequence contained in the first polynucleotide, the regulatory sequence contained in the second polynucleotide, or both regulatory sequences, contain a promoter functional in yeast cells and sensitive to the action of an inducing agent.

22. A method according to claim 21 characterised in that the inducible promoter functional in yeast cells is chosen from PGK1, ADH1, TDH3, LEU2 and TEF1.

23. A method according to claim 21 characterised in that the inducible promoter functional in yeast cells is chosen from CUP1, GAL1, MET3, MET25, MET28, SAM4 and PHO5.

24. A method according to claim 20, characterised in that the first polynucleotide contains the regulatory sequence GAL1, which activates the expression of the open reading frame coding for the fusion protein containing the polypeptide the IκBα polypeptide in the presence of glucose.

25. A method according to claim 20, characterised in that the second polynucleotide contains the regulatory sequence CUP1, which activates the expression of the open reading frame coding for a protein containing the polypeptide β-TrCP in the presence of copper sulphate.

26. A method according to claim 20, characterised in in that the recombinant yeast cells have the first and second polynucleotide in a form integrated into their genome.

27. A method according to claim 1, characterised in that the recombinant yeast cells have in their genome an inactivated form of one or several genes which controls the expression of transporter proteins in the plasma membrane.

28. A method according to claim 27, characterised in that the inactivated genes are chosen from genes PDR1 and PDR3.

29. An expression cassette functional in yeast cells containing a coding polynucleotide which includes an open reading frame encoding the fusion protein which contains the polypeptide the IκBα polypeptide and at least one first detectable protein, and a regulatory sequence functional in yeast cells which controls the expression of said open reading frame.

30. An expression cassette functional in yeast cells including a polynucleotide which contains an open reading frame encoding a protein containing the β-TrCP polypeptide and a regulatory sequence functional in yeast cells which controls the expression of said open reading frame.

31. An expression cassette according claim 29, characterised in that the regulatory sequence contained in said polynucleotide, the regulatory sequence contained in the second polynucleotide, or both regulatory sequences, contain a promoter functional in yeast cells and sensitive to the action of an inducing agent.

32. An expression cassette according to claim 31 characterised in that the inducible promoter functional in yeast cells is chosen from PGK1, TEF1, PHO5, MET3, MET28, CUP1, GAL1 and SAM4.

33. An expression vector characterised in that it contains an expression cassette according to claim 29.

34. An expression vector according to claim 33, characterised in that it is the vector pCSY226-NLS-IκBα.

35. An expression vector according to claim 33, characterised in that it is the vector pCSY226-NLS-β-TrCP.

36. A recombinant yeast strain containing, in a form integrated into its genome, (i) a first polynucleotide that contains an open reading frame coding for the fusion protein containing the polypeptide the IκBα polypeptide, and at least one first detectable protein, and a regulatory sequence functional in yeast cells which controls expression of said open reading frame; and
(ii) a second polynucleotide that contains an open reading frame coding for a protein containing the β-TrCP polypeptide and a regulatory sequence functional in yeast cells which controls expression of said open reading frame.

37. A recombinant yeast strain according to claim 36, characterised in that it consists of the yeast strain CYS135 deposited in the Collection Nationale de Cultures of microorganismes at the Institut Pasteur de Paris (CNCM) under accession number I-3187.

38. The tools or kit for screening agents modulating the ubiquitination of the IκBα protein by a functional ubiquitin ligase protein complex containing the β-TrCP protein, characterised in that it contains
(i) a first expression vector containing an expression cassette according to claim 29; and
(ii) a second expression vector containing an expression cassette functional in yeast cells including a polynucleotide which contains an open reading frame encoding a protein containing the β-TrCP polypeptide and a regulatory sequence functional in yeast cells which controls the expression of said open reading frame.

39. The tools or kit for screening agents modulating the ubiquitination of the IκBα protein by a functional ubiquitin ligase protein complex containing the β-TrCP protein, characterised in that it includes recombinant yeast cells containing, in a form integrated into their genome, respectively
(i) an expression cassette according to claim 29; and
(ii) an expression cassette functional in yeast cells including a polynucleotide which contains an open reading frame encoding a protein containing the β-TrCP polypeptide and a regulatory sequence functional in yeast cells which controls the expression of said open reading frame.

40. The tools or kit according to claim 39, characterised in that it contains recombinant yeast cells of the strain CYs135 deposited at the CNCM under accession number I-3187. _


SEQUENCE LISTING (26 sequences)

SEQ ID NO: 1 - DNA, 1719 bp, Artificial Sequence
  Description of Artificial Sequence Synthetic construct
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaactgcagagcccacctccaaaaaagaagagaaaggtcgaattgggcggatccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggctttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatga

SEQ ID NO: 2 - protein, 572 aa, Artificial Sequence [sequence omitted]

SEQ ID NO: 3 - DNA, 2583 bp, Artificial Sequence
  Description of Artificial Sequence Synthetic construct
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaactgcagagcccacctccaaaaaagaagagaaaggtcgaattgggcggatccatggacccggccgaggcggtgctgcaagagaaggcactcaagtttatgtgctctatgcccaggtctctgtggctgggctgctccagcctggcggacagcatgccttcgctgcgatgcctgtataacccagggactggcgcactcacagctttccagaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatattgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagatga

SEQ ID NO: 4 - protein, 860 aa, Artificial Sequence
  Description of Artificial Sequence Synthetic construct
MSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKLQSPPPKKKRKVELGGSMDPAEAVLQEKALKFMCSMPRSLWLGCSSLADSMPSLRCLYNPGTGALTAFQNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 5 - DNA, 21 bp, Simian virus 40
ccaaaaaagaagagaaaggtc

SEQ ID NO: 6 - DNA, 35 bp, Artificial Sequence
  Description of Artificial Sequence Synthetic primer
gctgggtaccttaataatcatattacatggcatta

SEQ ID NO: 7 - DNA, 34 bp, Artificial Sequence
  Description of Artificial Sequence Synthetic primer
ggcggaattctatagttttttctccttgacgtta

SEQ ID NO: 8 - DNA, 35 bp, Artificial Sequence
  Description of Artificial Sequence Synthetic primer
ggtcggaattcatgtctaaaggtgaagaattattc

SEQ ID NO: 9 - DNA, 46 bp, Artificial Sequence
  Description of Artificial Sequence Synthetic primer
ggcgggatccgcccgggctctgcagtttgtacaattcatccatacc

SEQ ID NO: 10 - DNA, 44 bp, Artificial Sequence
  Description of Artificial Sequence Synthetic primer
ggcggcggccgccaccgcggtgggcgaatttcttatgatttatg

SEQ ID NO: 11 - DNA, 30 bp, Artificial Sequence
  Description of Artificial Sequence Synthetic primer
ggcggagctctggaagaacgattacaacag

SEQ ID NO: 12 - DNA, 30 bp, Artificial Sequence
  Description of Artificial Sequence Synthetic primer
acctccaaaaaagaagagaaaggtcgaatt

SEQ ID NO: 13 - DNA, 31 bp, Artificial Sequence
  Description of Artificial Sequence Synthetic primer
ggcgggtaccgtgagtaaggaaagagtgagg

SEQ ID NO: 14 - DNA, 33 bp, Artificial Sequence
  Description of Artificial Sequence Synthetic primer
ggcggaattctgttttatatttgttgtaaaaag

SEQ ID NO: 15 - DNA, 33 bp, Artificial Sequence
  Description of Artificial Sequence Synthetic primer
ggcggaattcatggactacaaagaccatgacgg

SEQ ID NO: 16 - DNA, 46 bp, Artificial Sequence
  Description of Artificial Sequence Synthetic primer
ggcgggatccgcccgggctctgcagcttgtcatcgtcatccttgta

SEQ ID NO: 17 - protein, 9 aa, Artificial Sequence
  Description of Artificial Sequence Synthetic peptide
YPYDVPDYA

SEQ ID NO: 18 - protein, 8 aa, Artificial Sequence
  Description of Artificial Sequence Synthetic peptide
DYKDDDDK

SEQ ID NO: 19 - protein, 23 aa, Artificial Sequence
  Description of Artificial Sequence Synthetic peptide
MDYKDHDGDYKDHDIDYKDDDDK

SEQ ID NO: 20 - protein, 16 aa, Artificial Sequence
  Description of Artificial Sequence Synthetic peptide
KRPAATKKAGQAKKKK

SEQ ID NO: 21 - protein, 13 aa, Artificial Sequence
  Description of Artificial Sequence Synthetic peptide
MNKIPIKDLLNPQ

SEQ ID NO: 22 - protein, 19 aa, Artificial Sequence
  Description of Artificial Sequence Synthetic peptide
VRILESWFAKNIENPKLDT

SEQ ID NO: 23 - protein, 74 aa, Artificial Sequence
  Description of Artificial Sequence Synthetic peptide
MKLLSSIEQACDICRLKKLKCSKEKPKCAKCLKNNWECRYSPKTKRSPLTRAHLTEVESRLERLEQLFLLIFPR

SEQ ID NO: 24 - protein, 7 aa, Artificial Sequence
  Description of Artificial Sequence Synthetic peptide
PKKKRKV

SEQ ID NO: 25 - DNA, 30 bp, Artificial Sequence; identical to SEQ ID NO: 12
  Description of Artificial Sequence Synthetic primer

SEQ ID NO: 26 - DNA, 30 bp, Artificial Sequence
  Description of Artificial Sequence Synthetic primer
aattcgacctttctcttcttttttggaggt